(19) United States
(12) Patent Application Publication
(10) Pub. No.: US 2007/0134260 A1
(43) Pub. Date: Jun. 14, 2007

(54) Use of clusterin for the treatment and/or prevention of peripheral neurological diseases

(76) Inventors: Georg Feger, Thoiry (FR); Ursula Boschert, Troinex (CH); Yves Sagot, Beumont (FR); Ruben Papoian, Cincinnati, OH (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/550,775

(22) PCT Filed: Mar. 26, 2004
(86) PCT No.: PCT/EP04/50372

§ 371 Date: Jun. 30, 2006

(30) Foreign Application Priority Data
Mar. 28, 2003 (EP) 03100833.7

(51) Int. Cl. A61K 39/00

(52) U.S. Cl. 424/185.1

(57) ABSTRACT

The invention relates to the use of clusterin, or of an agonist of clusterin activity, for treatment or prevention of peripheral neurological diseases, The invention further relates to the use of a combination of clusterin and heparin, for treatment or prevention of peripheral neurological diseases.

### FIELD OF THE INVENTION

The present invention is generally in the field of neurological diseases of the peripheral nervous system. It relates to neuroprotection, nerve myelination and generation or re-generation of myelin producing cells. More specifically, the present invention relates to the use of clusterin, or of an agonist of clusterin activity, for the manufacture of a medicament for treatment and/or prevention of a peripheral neurological disease.

### BACKGROUND OF THE INVENTION

Peripheral neurological diseases are disorders relating to the peripheral nervous system (PNS) or the peripheral glia supporting the PNS. Peripheral neuropathies are among the most common peripheral neurological diseases.

Peripheral Neuropathy is a syndrome of sensory loss, muscle weakness and atrophy, decreased deep tendon reflexes, and vasomotor symptoms, alone or in any combination.

The disease may affect a single nerve (mononeuropathy), two or more nerves in separate areas (multiple mononeuropathy), or many nerves simultaneously (polyneuropathy). The axon may be primarily affected (e.g. in diabetes mellitus, Lyme disease, or uremia or with toxic agents) or the myelin sheath or Schwann cell (e.g. in acute or chronic inflammatory polyneuropathy, leukodystrophies, or Guillain-Barré syndrome). Damage to small unmyelinated and myelinated fibers results primarily in loss of temperature and pain sensation; damage to large myelinated fibers results in motor or proprioceptive defects. Some neuropathies (e.g. due to lead toxicity, dapsone use, tick bite, porphyria, or Guillain-Barré syndrome) primarily affect motor fibers; others (e.g. due to dorsal root ganglionitis of cancer, leprosy, AIDS, diabetes mellitus, or chronic pyridoxine intoxication) primarily affect the dorsal root ganglia or sensory fibers, producing sensory symptoms. Occasionally, cranial nerves are also involved (e.g. in Guillain-Barré syndrome, Lyme disease, diabetes mellitus, and diphtheria). Identifying the modalities involved helps determine the cause.

Trauma is the most common cause of a localized injury to a single nerve. Violent muscular activity or forcible overextension of a joint may produce a focal neuropathy, as may, repeated small traumas (e.g. tight gripping of small tools, excessive vibration from air hammers). Pressure or entrapment paralysis usually affects superficial nerves (ulnar, radial, peroneal) at bony prominences (e.g. during sound sleep or during anesthesia in thin or cachectic persons and often in alcoholics) or at narrow canals (e.g. in carpal tunnel syndrome). Pressure paralysis may also result from tumors, bony hyperostosis, casts, crutches, or prolonged cramped postures (e.g. in gardening). Hemorrhage into a nerve and exposure to cold or radiation may cause neuropathy. Mononeuropathy may result from direct tumor invasion.

Traumatic nerve injury of the PNS can be caused during surgery (e.g. surgical prostatectomy). In nerve-sparing prostatectomy, in order to avoid nerve damage, the practice is the stimulation of the cavernous nerve during surgery to identify the course of cavernous nerves and guide the surgeon in avoiding nerve damage (Klotz and Herschorn, 1998). Studies assessing the outcome of impotency following radical prostatectomy demonstrated 212 of 503 previously potent men (42%) suffered impotency when partial or complete resection of one or both cavernosal nerve(s). This impotency rate decreased to 24% when the nerves were left intact (Quinlan et al, 1991b; Quinlan et al., 1991a).

Multiple mononeuropathy is usually secondary to collagen vascular disorders (e.g. polyarteritis nodosa, SLE, Sjögren's syndrome, RA), sarcoidosis, metabolic diseases (e.g. diabetes, amyloidosis), or infectious diseases (e.g. Lyme disease, HIV infection). Microorganisms may cause multiple mononeuropathy by direct invasion of the nerve (e.g. in leprosy).

Polyneuropathy due to acute febrile diseases may result from a toxin (e.g. in diphtheria) or an autoimmune reaction (e.g. in Guillain-Barré syndrome); the polyneuropathy that sometimes follows immunizations is probably also autoimmune.

Toxic agents generally cause polyneuropathy but sometimes mononeuropathy. They include emetine, hexobarbital, barbital, chlorobutanol, sulfonamides, phenytoin, nitrofurantoin, the vinca alkaloids, heavy metals, carbon monoxide, triorthocresyl phosphate, orthodinitrophenol, many solvents, other industrial poisons, and certain AIDS drugs (e.g. zalcitabine, didanosine).

Nutritional deficiencies and metabolic disorders may result in polyneuropathy. B vitamin deficiency is often the cause (e.g. in alcoholism, beriberi, pernicious anemia, isonlazid-induced pyridoxine deficiency, malabsorption syndromes, and hyperemesis gravidarum). Polyneuropathy also occurs in hypothyroidism, porphyria, sarcoldosis, amyloldosis, and uremia. Diabetes mellitus can cause sensorimotor distal polyneuropathy (most common), multiple mononeuropathy, and focal mononeuropathy (e.g. of the oculomotor or abducens cranial nerves).

Malignancy may cause polyneuropathy via monoclonal gammopathy (multiple myeloma, lymphoma), amyloid invasion, or nutritional deficiencies or as a paraneoplastic syndrome.

Specific mononeuropathies: Single and multiple mononeuropathies are characterized by pain, weakness, and paresthesias in the distribution of the affected nerve. Multiple mononeuropathy is asymmetric; the nerves may be involved all at once or progressively. Extensive involvement of many nerves may simulate a polyneuropathy.

Ulnar nerve palsy is often caused by trauma to the nerve in the ulnar groove of the elbow by repeated leaning on the elbow or by asymmetric bone growth after a childhood fracture (tardy ulnar palsy). The ulnar nerve can also be compressed at is the cubital tunnel. Paresthesias and a sensory deficit in the 5th and medial half of the 4th fingers occur; the thumb adductor, 5th finger abductor, and interossel muscles are weak and atrophied. Severe chronic ulnar palsy produces a clawhand deformity. Nerve conduction studies can identify the site of the lesion. Conservative treatment should be attempted before surgical repair is attempted.

The carpal tunnel syndrome results from compression of the median nerve in the volar aspect of the wrist between the transverse superficial carpal ligament and the longitudinal tendons of forearm muscles that flex the hand. It may be unilateral or bilateral. The compression produces parestheslas in the radial-palmar aspect of the hand and pain in the wrist and palm; sometimes pain occurs proximally to the compression site in the forearm and shoulder. Pain may be more severe at night. A sensory deficit in the palmar aspect of the first three fingers may follow; the muscles that control thumb abduction and opposition may become weak and atrophied. This syndrome should be distinguished from C-6 root compression due to cervical radiculopathy.

Peroneal nerve palsy is usually caused by compression of the nerve against the lateral aspect of the fibular neck it is most common in emaciated bedridden patients and in thin persons who habitually cross their legs. Weakness of foot dorsiflexion and eversion (footdrop) occur. Occasionally, a sensory deficit occurs over the anterolateral aspect of the lower leg and dorsum of the foot or in the web space between the 1st and 2nd metatarsals. Treatment is usually conservative for compressive neuropathies (e.g. avoiding leg crossing). Incomplete neuropathles are usually followed clinically and usually improve spontaneously. If recovery does not occur, surgical exploration may be indicated.

Radial nerve palsy (Saturday night palsy) is caused by compression of the nerve against the humerus, e.g. as the arm is draped over the back of a chair during intoxication or deep sleep. Symptoms include weakness of wrist and finger extensors (wristdrop) and, occasionally, sensory loss over the dorsal aspect of the 1st dorsal interosseous muscle. Treatment is similar to that of compressive peroneal neuropathy.

Polyneuropathies are relatively symmetric, often affecting sensory, motor, and vasomotor fibers simultaneously. They may affect the axon cylinder or the myelin sheath and, in either form, may be acute (e.g. Guillain-Barré syndrome) or chronic (e.g. renal failure).

Polyneuropathy due to metabolic disorders (e.g. diabetes mellitus) or renal failure develops slowly, often over months or years. It frequently begins with sensory abnormalities in the lower extremities that are often more severe distally than proximally. Peripheral tingling, numbness, burning pain, or deficiencies in joint proprioception and vibratory sensation are often prominent. Pain is often worse at night and may be aggravated by touching the affected area or by temperature changes. In severe cases, there are objective signs of sensory loss, typically with stocking-and-glove distribution. Achilles and other deep tendon reflexes are diminished or absent. Painless ulcers on the digits or Charcot's joints may develop when sensory loss is profound. Sensory or proprioceptive deficits may lead to gait abnormalities. Motor involvement results in distal muscle weakness and atrophy. The autonomic nervous system may be additionally or selectively involved, leading to nocturnal diarrhea, urinary and fecal incontinence, impotence, or postural hypotension. Vasomotor symptoms vary. The skin may be paler and drier than normal, sometimes with dusky discoloration; sweating may be excessive. Trophic changes (smooth and shiny skin, pitted or ridged nails, osteoporosis) are common in severe, prolonged cases.

Nutritional polyneuropathy is common among alcoholics and the malnourished. A primary axonopathy may lead to secondary demyelination and axonal destruction in the longest and largest nerves. Whether the cause is deficiency of thiamine or another vitamin (e.g. pyridoxine, pantothenic acid, folic acid) is unclear. Neuropathy due to pyridoxine deficiency usually occurs only in persons taking isonlazid for TB; infants who are deficient or dependent on pyridoxine may have convulsions. Wasting and symmetric weakness of the distal extremities is usually insidious but can progress rapidly, sometimes accompanied by sensory loss, paresthesias, and pain. Aching, cramping, coldness, burning, and numbness in the calves and feet may be worsened by touch. Multiple vitamins may be given when etiology is obscure, but they have no proven benefit.

Uncommonly, an exclusively sensory polyneuropathy begins with peripheral pains and paresthesias and progresses centrally to a loss of all forms of sensation. It occurs as a remote effect of carcinoma (especially bronchogenic), after excessive pyridoxine ingestion (>0.5 g/day), and in amyloldosis, hypothyroidism, myeloma, and uremia. The pyridoxine-induced neuropathy resolves when pyridoxine is discontinued.

Hereditary neuropathies are classified as sensorimotor neuropathies or sensory neuropathies. Charcot-Marie-Tooth disease is the most common hereditary sensorimotor neuropathy. Less common sensorimotor neuropathies begin at birth and result in greater disability. In sensory neuropathies, which are rare, loss of distal pain and temperature sensation is more prominent than loss of vibratory and position sense. The main problem is pedal mutilation due to pain insensitivity, with frequent infections and osteomyelitis.

Hereditary motor and sensory neuropathy types I and II (Charcot -Marie-Tooth disease, peroneal muscular atrophy) is a relatively common, usually autosomal dominant disorder characterized by weakness and atrophy, primarily in peroneal and distal leg muscles. Patients may also have other degenerative diseases (e.g. Friedreich's ataxia) or a family history of them. Patients with type I present in middle childhood with footdrop and slowly progressive distal muscle atrophy, producing “stork legs.” Intrinsic muscle wasting in the hands begins later. Vibration, pain, and temperature sensation decreases in a stocking-glove pattern. Deep tendon reflexes are absent. High pedal arches or hammer toes may be the only signs in less affected family members who carry the disease. Nerve conduction velocities are slow, and distal latencles prolonged. Segmental demyelination and remyelination occur. Enlarged peripheral nerves may be palpated. The disease progresses slowly and does not affect life span. Type II disease evolves more slowly, with weakness usually developing later in life. Patients have relatively normal nerve conduction velocities but low amplitude evoked potentials. Biopsies show wallerian degeneration.

Hereditary motor and sensory neuropathy type III (hypertrophic interstitial neuropathy, Dejerine-Sottas disease), a rare autosomal recessive disorder, begins in childhood with progressive weakness and sensory loss and absent deep tendon reflexes. Initially, it resembles Charcot-Marie-Tooth disease, but motor weakness progresses at a faster rate. Demyelination and remyelination occur, producing enlarged peripheral nerves and onion bulbs seen on nerve biopsy.

The characteristic distribution of motor weakness, foot deformities, family history, and electrophysiologic abnormalities confirm the diagnosis. Genetic analysis is available, but no specific treatment Vocational counseling to prepare young patients for disease progression may be useful. Bracing helps correct footdrop; orthopedic surgery to stabilize the foot may help.

Spinal cord injuries account for the majority of hospital admissions for paraplegia and tetraplegia. Over 80% occur as a result of road accidents. Two main groups of injury are recognised clinically: open injuries and closed injuries.

Open injuries cause direct trauma of the spinal cord and nerve roots. Perforating injuries can cause extensive disruption and hemorrhage. Closed injuries account for most spinal injuries and are usually associated with a fracture/dislocation of the spinal column, which is usually demonstrable radiologically. Damage to the cord depends on the extent of the bony injuries and can be considered in two main stages: Primary damage, which are contusions, nerve fiber transections and hemorrhagic necrosis, and secondary damage, which are extradural heamatoma, infarction, infection and edema.

Late effects of cord damage include: ascending and descending anterograde degeneration of damaged nerve fibers, post-traumatic syringomelyia, and systemic effects of paraplegia, such as urinary tract and chest infections, pressure sores and muscle wasting.

Demyelination is linked to functional reduction or blockage in neural impulse conduction.

The multilamellar myelin sheath is a specialized domain of the glial cell plasma membrane, rich in lipid and low in protein. It serves to support axons and improve the efficiency of electrical signal conduction in the nervous system by preventing the charge from bleeding off into the surrounding tissue. The nodes of Ranvier are the sites in the sheath along the axon where saltatory conductance occurs.

The process of remyelination could work in concert with anti-inflammatory pathways to repair damage and protect axons from transection and death.

Schwann cells are peripheral glia cells providing a supportive role in the peripheral nervous system and belong to the satellite cells. Schwann cells wrap individually around the shaft of peripheral axons, forming a layer or myelin sheath along segments of the axon. Schwann cells are composed primarily of lipids or fats; the fat serves as an insulator thereby speeding the transmission rate of action potentials along the axon.

Schwann cells are also essential to the process of neuronal regeneration in the peripheral nervous system. When an axon is dying, the Schwann cells surrounding it aid in its digestion. This leaves an empty channel formed by successive Schwann cells, through which a new axon may grow from a severed end at a rate of 3-4 millimeters a day.

Neuropathies are usually selective as to the type of PNS neuron affected (e.g. sensory versus autonomic) and indeed also to the subtype of neurons (small versus large). Axotomy of peripheral nerves is the most commonly used animal model for appraising the neuroprotective effects of neurotrophic factors. Traumatic nerve injury, plexus lesions and root lesions are a serious complication of accidents. In addition, pressure on peripheral nerve that can cause myelin damage frequently seen in disorders such as carpal tunnel syndrome or is associated with spinal column orthopedic complications. Axotomy produces phenomena, like cell death, reduced axonal conduction velocity, and altered neurotransmitter levels in damaged neurons. Crush lesions allow for regeneration, an additional process of interest in relation to neuropathic states (McMahon and Priestley, 1995).

A fundamental question in cellular neurobiology is the regulation of nerve regeneration after injury or disease. Functional nerve regeneration requires not only axonal sprouting and elongation, but also new myelin synthesis. Remyelination is necessary for the restoration of normal nerve conduct ion and for protection of axons from new neurodegenerative immunologic attacks. The primary goal of research in neurodegenerative disorders is ultimately to develop interventions that prevent neuronal death, maintain neuronal phenotype and repair neuronal and myelin damage. Many studies have been devoted to the unraveling of molecular and cellular mechanisms responsible for the complete regeneration of axotomized spinal motor neurons (Fawcett and Keynes, 1990; Funakoshi et al., 1993). Injury-induced expression of neurotrophic factors and corresponding receptors may play an important role in the ability of nerve regeneration. Previous studies have shown a significant improvement of nerve regeneration with various peptides and non-peptides compounds like insulin-like growth factor (IGF-1), ACTH (Lewis et al., 1993; Strand et al., 1993), testosterone (Jones, 1993), SR 57746A (Fournier et al., 1993) and 4-Methylcatechol (Hanaoka et al., 1992: Kaechi et al., 1993).

Clusterin is an extracellular protein that is also known as Apolipoprotein J, SGP-2, TRPM-2 and SP40,40. It has a nearly ubiquitous tissue distribution and many names have been given to it according to the source where it was purified (reviewed in Trougakos and Gonos (Trougakos and Gonos, 2002), Jones and Jomary (Jones and Jomary, 2002)). Despite its ubiquitous expression and its relative abundance of serum (100 ug/ml) the genuine function of clusterin remains unraveled. Several biological roles of clusterin have been proposed among which the ability to inhibit complement cascade by binding C9 complement (Tschopp et al., 1993), a pro-apoptotic activity or an anti-apoptotic activity depending on animal models studied (Han et al., 2001; Wehrli et al., 2001), limitation of progression and more recently chaperone properties (Poon et al., 2002). A neuroprotective role of dusterin in Alzheimer's disease has also been suggested (Giannakopoulos et al., 1998). Its major form, a 75-80 kDa heterodimer is issued from a single transcript. The polypeptide chain is then cleaved proteolytically to remove the 22-mer secretory signal peptide and subsequently between residues 227/228 to generate two chains, alpha and beta, that are assembled by 5 cysteine-bonds located in the center of each chain. The polypeptide also contains glycosylation sites and nuclear localization signals sequences. Its degradation seems to be mediated by the endocytic receptor gp330/megalin/LRP2 a member of the low-density lipoprotein receptor family (Kounnas et al., 1995).

Heparin, refers to a highly acidic mucopolysaccharide formed of equal parts of sulfated D-glucosamine and D-glucuronic acid with sulfaminic bridges. The molecular weight ranges from six to twenty thousand. Heparin occurs in and is obtained from liver, lung, mast cells, etc., of vertebrates. Its function is unknown, but it is used to prevent blood clotting in vivo and vitro, in the form of many different salts (Medical Subject Headings (MESH), http:/www.nlm.nih.gov/mesh/meshhome.html). Heparin sodium (trade names: Lipo-Hepin and Liquaemin) is used as an anticoagulant in the treatment of thrombosis.

Low molecular weight heparins (LMWHs), heparin fractions, also exist. They have a molecular weight usually between 4000 and 6000 kD. These low-molecular-weight fractions are effective antithrombotic agents. Their administration reduces the risk of hemorrhage, they have a longer half-life, and their platelet interactions are reduced in comparison to unfractionated heparin. They also provide an effective prophylaxis against postoperative major pulmonary embolism (Medical Subject Headings (MESH), http:/www.nlm.nih.gov/mesh/meshhome.html). LMWHs can be e.g nadroparin, N-acetylheparin, ardeparin, certoparin, dalteparin, enoxaparin, reviparin, tinzaparin.

Other Heparins include Heparinoids. These are naturally occurring and synthetic highly-sulphated polysaccharides of similar structure. Hepardnoid preparations e.g. danaparoid sodium, have been used for a wide range of applications including as anticoagulants and anti-inflammatories and they have been claimed to have hypolipidemic properties (Martindale, The Extra Pharmacopoeia, 30th, p 232).

Interferons are a subclass of cytokines that exhibit anti-inflammatory, antiviral and anti-proliferative activity. On the basis of biochemical and immunological properties, the naturally-occurring human interferons are grouped into three classes: Interferon alpha (leukocyte), Interferon beta (fibroblast) and Interferon gamma (Immune). Alpha-interferon is currently approved in the United States and other countries for the treatment of hairy cell leukemia, venereal warts, Kaposi's Sarcoma (a cancer commonly afflicting patients suffering from Acquired Immune Deficiency Syndrome (AIDS)), and chronic non-A, non-B hepatitis.

Further, Interferons (IFNs) are glycoproteins produced by the body in response to a viral infection. They inhibit the multiplication of viruses in protected cells. Consisting of a lower molecular weight protein, IFNs are remarkably non-specific in their action, i.e. IFN induced by one virus is effective against a broad range of other viruses. They are however species-specific, i.e. IFN produced by one species will only stimulate antiviral activity in cells of the same or a closely related species. IFNs were the first group of cytokines to be exploited for their potential antitumour and antiviral activities.

The three major IFNs are referred to as IFN-α, IFN-β and IFN-γ. Such main kinds of IFNs were initially classified according to their cells of origin (leukocyte, fibroblast or T cell). However, it became clear that several types might be produced by one cell. Hence leukocyte IFN is now called IFN-α, fibroblast IFN is IFN-β and T cell IFN is IFN-γ. There is also a fourth type of IFN, lymphoblastoid IFN, produced in the “Namalwa” cell line (derived from Burkitt's lymphoma), which seems to produce a mixture of both leukocyte and fibroblast IFN.

The Interferon unit has been reported as a measure of IFN activity defined (somewhat arbitrarily) as the amount necessary to protect 50% of the cells against viral damage.

Every class of IFN contains several distinct types. IFN-β and IFN-γ are each the product of a single gene. The differences between individual types seem to be mainly due to variations in glycosylation.

IFNs-α are the most diverse group, containing about 15 types. There is a cluster of IFN-α genes on chromosome 9, containing at least 23 members, of which 15 are active and transcribed. Mature IFNs-α is not glycosylated.

IFNs-α and IFN-β are all the same length (165 or 166 amino acids) with similar biological activities. IFNs-γ are 146 amino acids in length, and resemble the α and β classes less closely. Only IFNs-γ can activate macrophages or induce the maturation of killer T cells. In effect, these new types of therapeutic agents can be called biologic response modifiers (BRMs), because they have an effect on the response of the organism to the tumour, affecting recognition via immunomodulation.

In particular, human fibroblast interferon (IFN-β) has antiviral activity and can also stimulate natural killer cells against neoplastic cells. It is a polypeptide of about 20,000 Da induced by viruses and double-stranded RNAs. From the nucleotide sequence of the gene for fibroblast interferon, cloned by recombinant DNA technology, Derynck et al. (Derynck et al., 1980) deduced the complete amino acid sequence of the protein. It is 166 amino acid long.

Shepard et al. (Shepard et al., 1981) described a mutation at base 842 (Cys→Tyr at position 141) that abolished its anti-viral activity, and a variant clone with a deletion of nucleotides 1119-1121.

Mark et al. (Mark et al, 1984) inserted an artificial mutation by replacing base 469 (T) with (A) causing an amino acid switch from Cys→Ser at position 17. The resulting IFN-β was reported to be as active as the ‘native’ IFN-β and stable during long-term storage (−70° C.).

The mechanisms by which IFNs exert their effects are not completely understood. However, in most cases they act by affecting the induction or transcription of certain genes, thus affecting the immune system. In vitro studies have shown that IFNs are capable of inducing or suppressing about 20 gene products.

Osteopontin (OPN) is a highly phosphorylated sialoprotein that is a prominent component of the mineralized extracellular matrices of bones and teeth. OPN is characterized by the presence of a polyaspartic acid sequence and sites of Ser/Thr phosphorylation that mediate hydroxyapatite binding, and a highly conserved RGD motif that mediates cell attachment/signalling. Osteopontin inhibitors have been described said to be useful for treatment of infections, immune disorders and diseases, autoimmune disorders, including MS, various immunodeficiencies, and cancer, WO00/63241. The use of Osteopontin or of an agonist of osteopontin activity, is claimed in WO02/92122 for the manufacture of a medicament for the treatment and/or prevention of a neurologic disease.

Bonnard A et al, observed an increase of clusterin mRNA expression at the lesion site following rat sciatic nerve crush (Bonnard et al., 1997).

The treatement of PNS diseases with clusterin has not yet been considered in the art.

### SUMMARY OF THE INVENTION

It is the object of the present invention to provide novel means for the treatment and/or prevention of peripheral neurological diseases.

The invention is based on the finding that the protein clusterin has a beneficial effect in an animal model of peripheral neuropathy.

Therefore, the present invention relates to the use of clusterin, or of an agonist of clusterin activity, in a peripheral neurological disease, such as traumatic nerve injury of the peripheral nervous system (PNS), and peripheral neuropathies.

The use of nucleic acid molecules, and expression vectors comprising clusterin, and of cells expressing clusterin, for treatment and/or prevention of a peripheral neurological disease is also within the present invention.

The invention further provides pharmaceutical compositions comprising clusterin and heparin or an interferon or osteopontin, optionally together with one or more pharmaceutically acceptable excipients.

In a second aspect of the invention, clusterin may be used in combination with Heparin, an interferon or osteopontin for treatment and/or prevention of peripheral neurological diseases.

### BRIEF DESCRIPTION OF THE DRAWINGS

FIG. 1 schematically depicts the structure of clusterin (based on Rosenberg and Silkensen, 1995). (A) is the precursor polypeptide, (B) is a representation of the mature polypeptide, which is a heterodimeric glycoprotein of 75-80 kDa formed by an α (34-36 kDa) and β (36-39 kDa) chain linked in antiparallel by 5 disulfide bridges near their centers, (C) shows the sequence of human clusterin precursor.

FIG. 2 shows the body weight in grams (g) of neuropathic mice induced by sciatic nerve crush treated with vehicle (open circle), 300 μg/kg (closed triangle) or 1 mg/kg of mclusterin (closed losange) administered intraperitoneally (i.p.). Control: healthy mice (closed square).

FIG. 3 shows the amplitude in millivolt (mV) of the compound muscle action potential in neuropathic mice treated with vehicle, 300 μg/kg or 1 mg/kg i.p. of mclusterin, 0.01 μg/kg of a positive control compound (4-MC) or 100 μg/kg subcutaneous (s.c.) of osteopontin. Control: sham operated mice.

FIG. 4 shows the latency in milliseconds (ms) of the compound muscle action potential in neuropathic mice treated with vehicle, 300 μg/kg or 1 mg/kg i.p. of mclusterin, 0.01 μg/kg of a positive control compound (4-MC) or 100 μg/kg s.c. of osteopontin Control: sham operated mice.

FIG. 5 shows the duration in milliseconds (ms) of the compound muscle action potential in the neuropathic mice treated with vehicle, 300 μg/kg or 1 mg/kg i.p. of mclusterin, 0.01 μg/kg of a positive control compound (4-MC) or 100 μg/kg s.c. of osteopontin. Control: sham operated mice.

FIG. 6 shows the percentage of degenerated fibers in the neuropathic mice treated with vehicle, 300 μg/kg, or 1mg/kg i.p. of mclusterin. Control: sham operated mice.

FIG. 7 shows the percentage of non-degenerated fibers in the neuropathic mice treated with vehicle, 300 μg/kg, or 1 mg/kg of mclusterin. Control: sham operated mice.

FIG. 8 shows the amplitude in millivolt (mV) of the compound muscle action potential in neuropathic mice treated with vehicle, 100 μg/kg, 300 μg/kg or 1000 μg/kg s.c. of recombinant hclusterin and 30 μg/kg s.c. of a positive control (recombinant hIL-6. Recording was performed 1, 2, 3, or 4 weeks after sciatic nerve injury. Data are expressed as mean total amplitude in mV±standard error; n=6 mice per group, #p<0.01, *p<0.05,**p<0.01.

FIG. 9 shows the choline acetyl transferase (ChAT) activity in cpm (count per minute) by microgram of protein (cpm/μg protein) in gastrocnemius muscle of contralateral side (a) and ipsilateral side (b) of neuropathic mice treated s.c. for 4 weeks with vehicle, 30 μg/kg of recombinant human IL-6 or 100 μg/kg, 300 μg/kg and 1000 μg/kg of recombinant hClusterin. n=6 mice per group. #p<0.1.

FIG. 10 shows the neurofilaments-high molecular weight form (NF-H) content, in nanogram per microgram of protein (ng of NF-H/mg proteins), in (a) the contralateral sciatic nerve and (b) the proximal (above the crush) and (c) distal (below the crush) parts of ipsilateral sciatic nerve after four weeks of treatment with vehicle, 30 μg/kg of recombinant human IL-6 or 100 μg/kg, 300 μg/kg and 1000 μg/kg s.c. of recombinant hclusterin. n=6 mice per group. *p<0.01.

FIG. 11 shows the Myelin Basic Protein (MBP) content, in picogram per microgram of protein (pg MBP/μg total proteins), of organotypic hippocampal slices treated with 1 μg/ml of recombinant mclusterin at 3, 6 and 10 days of treatment (T3, T6 and T10) corresponding to 10, 13 and 17 days in vitro (DIV). Control group received normal medium (60% MEM, 25% HBSS, 25% horse serum). Similar results are obtained when recombinant human clusterin from HEK or from CHO cells is used (data not shown). Data are expressed as mean total MBP±standard error; exp=2, n=12 per group, p<0.001***.

FIG. 12 shows the MBP content in picogram per microgram of protein (pg MBP/μg tot prot) of organotypic hippocampal slices, treated with 10, 100 and 1000 ng/ml of recombinant mclusterin, after specific demyelination induced by anti-MOG (anti-myelin oligodendrocyte glycoprotein) antibodies in combination with complement (IgG anti-MOG+complement) or by non-relevant isotype matching immunoglobulin IgG and complement (IgG control+complement). As a control, an untreated group received normal medium (50% MEM, 25% HBSS, 25% horse serum). mClusterin was applied at 21 DIV (Day in vitro), 24 hours before the addition of the antibodies and at the time of treatment exp=3, n=15, *p<0.05, **p<0.01, ***p<0.001.

Similar results are obtained when recombinant human clusterin from HEK or from CHO cells is used (data not shown).

FIG. 13 shows the serum concentration of hclusterin in nanogram per milliliter (ng/ml) detected by ELISA, 5 or 30 minutes after intravenous (i.v.) injection of recombinant hclusterin (300 μg/kg) in the presence or in the absence of heparin (7500 U/kg).

A. Heparin administered 5 minutes before clusterin (heparin injected before clusterin) or concomitantly to clusterin (clusterin mixed with heparin). As a control mice were injected with clusterin alone (clusterin) and the blood was collected in a tube +/− heparin (clusterin collected in Heparin). n=3 mice/group; ***p<0.005.

B. Effect of Heparin (7500 U/kg) administration prior to blood collection. Group 1: Heparin administered 5 min before clusterin (1 mg/kg). Group 2: Heparin injected 28 min after clusterin (1 mg/kg). Group 3: clusterin (1 mg/kg) alone. a: Anova single factor test against group 1. b: Anova single factor test against group 2. N=4 mice/group, # p<0.1, *p<0.05, **p<0.01. Similar results were obtained with N-acetylheparin administration (data not shown).

### DETAILED DESCRIPTION OF THE INVENTION

In the frame of the present invention, it has been found that administration of clusterin has a beneficial effect in an in vivo animal model of peripheral neurological diseases. In a murine model of sciatic nerve crush induced neuropathy, all physiologic and morphologic parameters relating to nerve regeneration, integrity and vitality were positively influenced by administration of clusterin.

The invention therefore relates to the use of clusterin, an isoform, mutein, fused protein, functional derivative, active fraction, circularly permutated derivative, or salt thereof, or of an agonist of clusterin activity, for the manufacture of a medicament for treatment and/or prevention of peripheral neurological diseases.

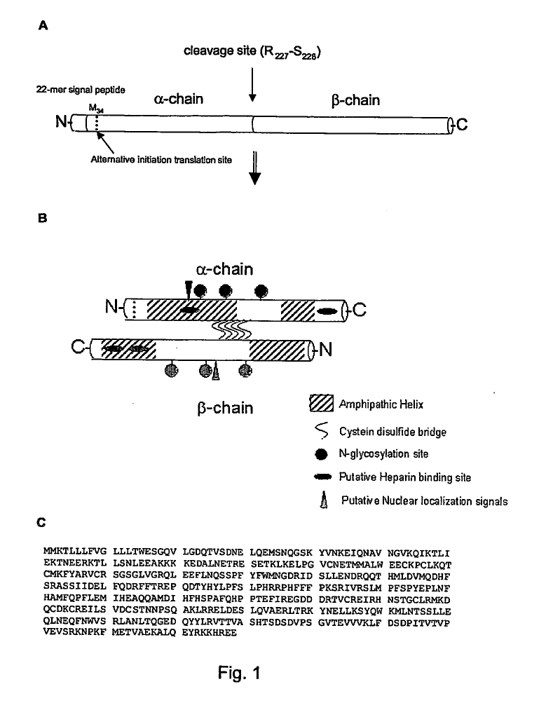

The term “clusterin”, as used herein, relates to full-length mature human clusterin, or to any of the clusterin subunits, or a fragment thereof. The sequence of human clusterin is reported herein as SEQ ID NO: 1 of the annexed sequence listing, and in FIG. 1C of the annexed drawings. The term “clusterin”, as used herein, further relates to any clusterin derived from animals, such as murine, bovine, porcine, feline or ovine clusterin, as long as there is sufficient identity in order to maintain clusterin activity, and as long as the resulting molecule will not be immunogenic in humans.

The term “clusterin”, as used herein, further relates to biologically active muteins and fragments, such as the naturally occurring alpha and beta subunit of clusterin.

The term “clusterin”, as used herein, further encompasses isoforms, muteins, fused proteins, functional derivatives, active fractions or fragments, or circularly permutated derivatives, or salts thereof. These isoforms, muteins, fused proteins or functional derivatives, active fractions or fragments, or circularly permutated derivatives retain the biological activity of clusterin. Preferably, they have a biological activity, which is imp roved as compared to wild type clusterin.

The term “agonist of clusterin activity”, as used herein, relates to a molecule stimulating or mimicking clusterin activities, such as agonistic antibodies of a clusterin receptor, or small molecular weight agonis is activating signaling through a clusterin receptor. A clusterin receptor maybe e.g. gp330/megalin/LRP2 (Kounnas et al., 1995). Any agonist, stimulator or enhancer, of such a receptor is encompassed by the term “agonist of clusterin activity”, as used herein.

The term “agonist of clusterin activity”, as used herein, further refers to agents enhancing clusterin mediated activities, such as small molecular weight compounds mimicking the clusterin activity.

The terms “treating” and “preventing”, as used here in, should be understood as preventing, inhibiting, attenuating, ameliorating or reversing one or more symptoms or cause(s) of peripheral neurological diseases, as well as symptoms, diseases or complications accompanying peripheral neurological disease. When “treating” peripheral neurological disease, the substances according to the invention are given after onset of the disease, “prevention” relates to administration of the substances before signs of disease can be noted in the patient.

The term “peripheral neurological diseases”, as used herein encompasses all known peripheral neurological diseases or disorders, or injuries of the PNS, including those described in detail in the “Background of the Invention”.

Peripheral neurological diseases comprise disorders linked to dysfunction of the PNS, such as diseases related to neurotransmission, nerve trauma, PNS infections, demyelinating diseases of the PNS, or neuropathies of the PNS.

Preferably, the peripheral neurological diseases of the invention are selected from the group consisting of traumatic nerve injury of the peripheral nervous system, demyelinating diseases of the PNS, and peripheral neurodegenerative diseases and peripheral neuropathies.

Traumatic nerve injury may concern the PNS as described in the “Background of the invention” above.

Peripheral neuropathy may be related to a syndrome of sensory loss, muscle weakness and atrophy, decreased deep tendon reflexes, and vasomotor symptoms, alone or in any combination. They may e.g. be due to alcoholism, diabetes or chemotherapeutic treatment.

Neuropathy may affect a single nerve (mononeuropathy), two or more nerves in separate areas (multiple mononeuropathy), or many nerves simultaneously (polyneuropathy). The axon may be primarily affected (e.g. in diabetes mellitus, Lyme disease, or uremia or with toxic agents), or the myelin sheath or Schwann cell (e.g. in acute or chronic inflammatory polyneuropathy, leukodystrophies, or Guillain-Barré syndrome). Further neuropathies, which may be treated in accordance with the present invention, may e.g. be due to lead toxicity, dapsone use, tick bite, porphyria, or Guillain-Barré syndrome, and they may primarily affect motor fibers. Others, such as those due to dorsal root ganglionitis of cancer, leprosy, AIDS, diabetes mellitus, or chronic pyridoxine intoxication, may primarily affect the dorsal root ganglia or sensory fibers, producing sensory symptoms. Cranial nerves may also be involved, such as e.g. in Guillain-Barré syndrome, Lyme disease, diabetes mellitus, and diphtheria.

Further peripheral neurological disorders comprise neuropathies with abnormal myelination, such as the ones listed in the “Background of the invention” above, as well as carpal tunnel syndrome. Traumatic nerve injury may be accompanied by spinal column orthopedic complications, and those are also within the diseases in accordance with the present invention.

Peripheral neurological disorders may further be due to congenital metabolic disorders. In a preferred embodiment of the invention, the peripheral neurological disease is therefore due to a congenital metabolic deficit.

In a further preferred embodiment, the peripheral neurological disease is a peripheral neuropathy, most preferably diabetic neuropathy. Chemotherapy associated neuropathies are also preferred in accordance with the present invention.

The term “diabetic neuropathy” relates to any form of diabetic neuropathy, or to one or more symptom(s) or disorder(s) accompanying or caused by diabetic neuropathy, or complications of diabetes affecting nerves as described in detail in the “Background of the invention” above. Diabetic neuropathy may be a polyneuropathy. In diabetic polyneuropathy, many nerves are simultaneously affected. The diabetic neuropathy may also be a mononeuropathy. In focal mononeuropathy, for instance, the disease affects a single nerve, such as the oculomotor or abducens cranial nerve. It may also be multiple mononeuropathy when two or more nerves are affected in separate areas.

In yet a further preferred embodiment, the peripheral neurological disorder is a demyelinating disease of the peripheral nervous system (PNS). The latter comprise diseases such as chronic inflammatory demyelinating polyradiculoneuropathy (CIDP) and acute, monophasic disorders, such as the inflammatory demyelinating polyradiculoneuropathy termed Guillain-Barré syndrome (GBS).

Preferably, the clusterin is selected from a peptide, a polypeptide or a protein selected from the group consisting of:
a) A polypeptide comprising SEQ ID NO: 1;
b) A polypeptide comprising amino acids 23 to 449 of SEQ ID NO: 1;
c) A polypeptide comprising amino acids 35 to 449 of SEQ ID NO: 1,
d) A polypeptide comprising amino acids 23 to 227 of SEQ ID NO: 1;
e) A polypeptide comprising amino acids 35 to 227 of SEQ ID NO: 1;
f) A polypeptide comprising amino acids 228 to 449 of SEQ ID NO: 1;
g) A mutein of any of (a) to (f), wherein the amino acid sequence has at least 40% or 50% or 60% or 70% or 80% or 90% identity to at least one of the sequences in (a) to (f);
h) A mutein of any of (a) to (f) which is encoded by a DNA sequence which hybridizes to the complement of the native DNA sequence encoding any of (a) to (f) under moderately stringent conditions or under highly stringent conditions;
i) A mutein of any of (a) to (f) wherein any changes in the amino acid sequence are conservative amino acid substitutions to the amino acid sequences in (a) to (f);
j) a salt or an isoform, fused protein, functional derivative, active fraction or circularly permutated derivative of any of (a) to (f).

Active fractions or fragments may comprise any portion or domain of clusterin, such as the alpha chain or the beta chain separated, or linked to each other e.g. via di-sulfide bridges, directly fused, or fused via an appropriate linker. Active fractions also comprise differentially glycosylated or sialylated forms of clusterin.

The person skilled in the art will appreciate that even smaller portions of clusterin or its two subunits may be enough to exert its function, such as an active peptide comprising the essential amino acid residues required for clusterin function.

The person skilled in the art will further appreciate that muteins, salts, isoforms, fused proteins, functional derivatives of clusterin, active fractions or circularly permutated derivatives of clusterin, will retain a similar, or even better, biological activity of clusterin. The biological activity of clusterin and muteins, isoforms, fused proteins or functional derivatives, active fractions or fragments, circularly permutated derivatives, or salts thereof, may be measured in a co-culturing assay.

Preferred active fractions have an activity which is equal or better than the activity of full-length clusterin, or which have further advantages, such as a better stability or a lower toxicity or immunogenicity, or they are easier to produce in large quantities, or easier to purify. The person skilled in the art will appreciate that muteins, active fragments and functional derivatives can be generated by cloning the corresponding cDNA in appropriate plasmids and testing them in the co-culturing assay, as mentioned above.

The proteins according to the present invention may be glycosylated or non-glycosylated, they may be derived from natural sources, such as body fluids, or they may preferably be produced recombinantly. Recombinant expression may be carried out in prokaryotic expression systems such as E. coli, or in eukaryotic, such as insect cells, and preferably in mammalian expression systems, such as CHO-cells or HEK-cells.

As used herein the term “muteins” refers to analogs of clusterin, in which one or more of the amino acid residues of a natural clusterin are replaced by different amino acid residues, or are deleted, or one or more amino acid residues are added to the natural sequence of clusterin, without changing considerably the activity of the resulting products as compared with the wild-type clusterin. These muteins are prepared by known synthesis and/or by site-directed mutagenesis techniques, or any other known technique suitable therefore.

Muteins of clusterin, which can be used in accordance with the present invention, or nucleic acid coding thereof, include a finite set of substantially corresponding sequences as substitution peptides or polynucleotides which can be routinely obtained by one of ordinary skill in the art, without undue experimentation, based on the teachings and guidance presented herein.

Muteins in accordance with the present invention include proteins encoded by a nucleic acid, such as DNA or RNA, which hybridizes to DNA or RNA, which encodes clusterin, in accordance with the present invention, under moderately or highly stringent conditions. The term “stringent conditions” refers to hybridization and subsequent washing conditions, which those of ordinary skill in the art conventionally refer to as “stringent”. See Ausubel et al., Current Protocols in Molecular Biology, supra, interscience, N.Y., §§6.3 and 6.4 (1987, 1992), and Sambrook et al. (Sambrook J. C., Fritsch, E. F., and Maniatis, T. (1989) Molecular Cloning: A Laboratory Manual, Cold Spring Harbor Laboratory Press, Cold Spring Harbor, N.Y.).

Without limitation, examples of stringent conditions include washing conditions 12-20° C. below the calculated Tm of the hybrid under study in, e.g., 2×SSC and 0.5% SDS for 5 minutes, 2×SSC and 0.1% SDS for 15 minutes; 0.1×SSC and 0.5% SDS at 37° C. for 30-60 minutes and then, a 0 1×SSC and 0.5% SDS at 68° C. for 30-60 minutes. Those of ordinary skill in this art understand that stringency conditions also depend on the length of the DNA sequences, oligonucleotide probes (such as 1040 bases) or mixed oligonucleotide probes. If mixed probes are used, it is preferable to use tetramethyl ammonium chloride (TMAC) instead of SSC. See Ausubel, supra.

In a preferred embodiment, any such mutein has at least 40% identity or homology with the sequence of SEQ ID NO: 1 of the annexed sequence listing. More preferably, it has at least 50%, at least 60%, at least 70%, at least 80% or, most preferably, at least 90% identity or homology thereto.

Identity reflects a relationship between two or more polypeptide sequences or two or more polynucleotide sequences, determined by comparing the sequences. In general, identity refers to an exact nucleotide to nucleotide or amino acid to amino acid correspondence of the two polynucleotides or two polypeptide sequences, respectively, over the length of the sequences being compared.

For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called global alignment), that is particularly suitable for sequences of the same or very similar length, or over shorter, defined lengths (so-called local alignment), that is more suitable for sequences of unequal length.

Methods for comparing the identity and homology of two or more sequences are well known in the art Thus for instance, programs available in the Wisconsin Sequence Analysis Package, version 9.1 (Devereux et al., 1984), for example the programs BESTFIT and GAP, may be used to determine the % identity between two polynucleotides and the % identity and the % homology between two polypeptide sequences. BESTFIT uses the “local homology” algorithm of Smith and Waterman (Smith and Waterman, 1981) and finds the best single region of similarity between two sequences. Other programs for determining identity and/or similarity between sequences are also known in the art, for instance the BLAST family of programs (Altschul et al., 1990; Altschul et al., 1997). accessible through the home page of the NCBI at www.ncbi.nlm.nih.gov) and FASTA (Pearson, 1990: Pearson and Lipman, 1988).

Preferred changes for muteins in accordance with the present invention are what are known as “conservative” substitutions. Conservative amino acid substitutions of clusterin polypeptides, may include synonymous amino acids within a group which have sufficiently similar physicochemical properties that substitution between members of the group will preserve the biological function of the molecule (Grantham, 1974). It is clear that insertions and deletions of amino acids may also be made in the above-defined sequences without altering their function, particularly if the insertions or deletions only involve a few amino acids, e.g. under thirty, and preferably under ten, and do not remove or displace amino acids which are critical to a functional conformation, e.g. cysteine residues. Proteins and muteins produced by such deletions and/or insertions come within the purview of the present invention.

Preferably, the synonymous amino acid groups are those defined in Table I. More preferably, the synonymous amino acid groups are those defined in Table II; and most preferably the synonymous amino acid groups are those defined in Table III.

TABLE I
Preferred Groups of Synonymous Amino Acids

| Amino Acid | Synonymous Group |
|---|---|
| Ser | Ser, Thr, Gly, Asn |
| Arg | Arg, Gln, Lys, Glu, His |
| Leu | Ile, Phe, Tyr, Met, Val, Leu |
| Pro | Gly, Ala, Thr, Pro |
| Thr | Pro, Ser, Ala, Gly, His, Gln, Thr |
| Ala | Gly, Thr, Pro, Ala |
| Val | Met, Tyr, Phe, Ile, Leu, Val |
| Gly | Ala, Thr, Pro, Ser, Gly |
| Ile | Met, Tyr, Phe, Val, Leu, Ile |
| Phe | Trp, Met, Tyr, Ile, Val, Leu, Phe |
| Tyr | Trp, Met, Phe, Ile, Val, Leu, Tyr |
| Cys | Ser, Thr, Cys |
| His | Glu, Lys, Gln, Thr, Arg, His |
| Gln | Glu, Lys, Asn, His, Thr, Arg, Gln |
| Asn | Gln, Asp, Ser, Asn |
| Lys | Glu, Gln, His, Arg, Lys |
| Asp | Glu, Asn, Asp |
| Glu | Asp, Lys, Asn, Gln, His, Arg, Glu |
| Met | Phe, Ile, Val, Leu, Met |
| Trp | Trp |

TABLE II
More Preferred Groups of Synonymous Amino Acids

| Amino Acid | Synonymous Group |
|---|---|
| Ser | Ser |
| Arg | His, Lys, Arg |
| Leu | Leu, Ile, Phe, Met |
| Pro | Ala, Pro |
| Thr | Thr |
| Ala | Pro, Ala |
| Val | Val, Met, Ile |
| Gly | Gly |
| Ile | Ile, Met, Phe, Val, Leu |
| Phe | Met, Tyr, Ile, Leu, Phe |
| Tyr | Phe, Tyr |
| Cys | Cys, Ser |
| His | His, Gln, Arg |
| Gln | Glu, Gln, His |
| Asn | Asp, Asn |
| Lys | Lys, Arg |
| Asp | Asp, Asn |
| Glu | Glu, Gln |
| Met | Met, Phe, Ile, Val, Leu |
| Trp | Trp |

TABLE III
Most Preferred Groups of Synonymous Amino Acids

| Amino Acid | Synonymous Group |
|---|---|
| Ser | Ser |
| Arg | Arg |
| Leu | Leu, Ile, Met |
| Pro | Pro |
| Thr | Thr |
| Ala | Ala |
| Val | Val |
| Gly | Gly |
| Ile | Ile, Met, Leu |
| Phe | Phe |
| Tyr | Tyr |
| Cys | Cys, Ser |
| His | His |
| Gln | Gln |
| Asn | Asn |
| Lys | Lys |
| Asp | Asp |
| Glu | Glu |
| Met | Met, Ile, Leu |
| Trp | Met |

Examples of production of amino acid substitutions in proteins which can be used for obtaining muteins of clusterin, polypeptides or proteins, for use in the present invention include any known method steps, such as presented in U.S. Pat. Nos. 4,959,314, 4,588,585 and 4,737,462, to Mark et al. U.S. Pat. No. 5,116,943 to Koths et al., U.S. Pat. No. 4,965,195 to Namen et al; U.S. Pat. No. 4,879,111 to Chong et al, and U.S. Pat. No. 5,017,691 to Lee et al; and lysine substituted proteins presented in U.S. Pat. No. 4,904,584 (Shaw et al).

The term “fused protein” refers to a polypeptide comprising clusterin, or a mutein or fragment thereof, fused with another protein, which e.g. has an extended residence time in body fluids. Clusterin may thus be fused to another protein, polypeptide or the like, e.g. an immunoglobulin or a fragment thereof. Immunoglobulin Fc portions are particularly suitable for production of di- or mulitmeric Ig fusion proteins. The alpha- and beta-chain of clusterin may e.g. be linked to portions of an immunoglobulin in such a way as to produce the alpha- and beta-chain of clusterin dimerized by the Ig Fc portion.

“Functional derivatives” as used herein, cover derivatives of clusterin, and their muteins and fused proteins, which may be prepared from the functional groups which occur as side chains on the residues or the N- or C-terminal groups, by means known in the art, and are included in the invention as long as they remain pharmaceutically acceptable, i.e. they do not destroy the activity of the protein which is substantially similar to the activity of clusterin, and do not confer toxic properties on compositions containing it.

These derivatives may, for example, include polyethylene glycol side-chains, which may mask antigenic sites and extend the residence of clusterin in body fluids. Other derivatives include aliphatic esters of the carboxyl groups, amides of the carboxyl groups by reaction with ammonia or with primary or secondary amines, N-acyl derivatives of free amino groups of the amino acid residues formed with acyl moieties (e.g. alkanoyl or carbocyclic aroyl groups) or O-acyl derivatives of free hydroxyl groups (for example that of seryl or threonyl residues) formed with acyl moieties.

As “active fractions” of clusterin, muteins and fused proteins, the present invention covers any fragment or precursors of the polypeptide chain of the protein molecule alone or together with associated molecules or residues linked thereto, e.g. sugar or phosphate residues, or aggregates of the protein molecule or the sugar residues by themselves, provided said fraction has substantially similar activity to clusterin.

The term “salts” herein refers to both salts of carboxyl groups and to acid addition salts of amino groups of clusterin molecule or analogs thereof. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids, such as, for example, hydrochloric acid or sulfuric acid, and salts with organic acids, such as, for example, acetic acid or oxalic acid. Of course, any such salts must retain the biological activity of clusterin relevant to the present invention, i.e., neuroprotective effect in a peripheral neurological disease.

Functional derivatives of clusterin may be conjugated to polymers in order to improve the properties of the protein, such as the stability, half-life, bioavailability, tolerance by the human body, or immunogenicity. To achieve this goal, clusterin may be linked e.g. to Polyethlyenglycol (PEG). PEGylation may be carried out by known methods, described in WO 92,13095, for example.

Therefore, in a preferred embodiment of the present invention, clusterin is PEGylated.

In a further preferred embodiment of the invention, the fused protein comprises an immunoglobulin (Ig) fusion. The fusion may be direct, or via a short linker peptide which can be as short as 1 to 3 amino acid residues in length or longer, for example, 13 amino acid residues in length. Said linker may be a tripeptide of the sequence E-F-M (Glu-Phe-Met), for example, or a 13-amino acid linker sequence comprising Glu-Phe-Gly-Ala-Gly-Leu-VaWLeuGly-Gly-Gln-Phe-Met introduced between clusterin sequence and the immunoglobulin sequence, for instance. The resulting fusion protein has improved properties, such as an extended residence time in body fluids (half-life), or an increased specific activity, increased is expression level. The Ig fusion may also facilitate purification of the fused protein.

In a yet another preferred embodiment, clusterin or one or both subunits are fused to the constant region of an Ig molecule. Preferably, it is fused to heavy chain regions, like the CH2 and CH3 domains of human IgG1, for example. Other isoforms of Ig molecules are also suitable for the generation of fusion proteins according to the present invention, such as isoforms IgG₂ or IgG₄, or other Ig classes, like IgM, for example. Fusion proteins may be monomeric or multimeric, hetero- or homomultimeric. The immunoglobulin portion of the fused protein may be further modified in a way as to not activate complement binding or the complement cascade or bind to Fc-receptors.

The invention further relates to the use of a combination of clusterin and an immunosuppressive agent for the manufacture of a medicament for treatment and/or prevention of peripheral neurological disorders, for simultaneous, sequential or separate use. Immunosuppressive agents may be steroids, methotrexate, cyclophosphamide, anti-eukocyte antibodies (such as CAMPATH-1), and the like.

The invention further relates to the combination of clusterin and IL6.

Heparin administration has been shown to greatly improve clusterin bio-availability, therefore the invention further relates to the use of a combination of clusterin and heparin for the manufacture of a medicament for treatment and/or prevention of peripheral neurological disorders, for simultaneous, sequential, or separate use.

“Heparin”, as used herein, refers to all heparins and heparinoids known in the art such as the one described in the “Background of the invention” e.g. low molecular weight heparins (LMWHs).

The invention further relates to the use of a combination of clusterin and an interferon for the manufacture of a medicament for treatment and/or prevention of peripheral neurological disorders, for simultaneous, sequential, or separate use.

The term “interferon”, as used in the present patent application, is intended to include any molecule defined as such in the literature, comprising for example any kinds of IFNs mentioned in the above section “Background of the invention”. The interferon may preferably be human, but also derived from other species, as long as the biological activity is similar to human interferons, and the molecule is not immunogenic in man.

In particular, any kinds of IFN-α, IFN-β and IFN-γ are included in the above definition. IFN-β is the preferred IFN according to the present invention.

The term “interferon-beta (IFN-β)”, as used in the present invention, is intended to include human fibroblast interferon, as obtained by isolation from biological fluids or as obtained by DNA recombinant techniques from prokaryotic or eukaryotic host cells as well as its salts, functional derivatives, variants, analogs and fragments.

Interferons may also be conjugated to polymers in order to improve the stability of the proteins. A conjugate between Interferon β and the polyol polyethlyenglycol (PEG) has been described in WO99/55377, for instance.

In another preferred embodiment of the invention, the interferon is Interferon-β (IFN-β), and more preferably IFN-β1a.

Clusterin is preferably used simultaneously, sequentially, or separately with the interferon.

The invention further relates to the use of a combination of clusterin and osteopontin for the manufacture of a medicament for treatment and/or prevention of peripheral neurological disorders, for simultaneous, sequential, or separate use.

“Osteopontin”, as used herein, encompasses also muteins, fragments, active fractions and functional derivatives of osteopontin. These proteins are described e.g. in WO 02/092122.

In a preferred embodiment of the present invention, clusterin is used in an amount of about 0.001 to 100 mg/kg of body weight, or about 1 to 10 mg/kg of body weight or about 5 mg/kg of body weight.

The invention further relates to the use of a nucleic acid molecule for manufacture of a medicament for the treatment and/or prevention of a peripheral neurological disease, wherein the nucleic acid molecule comprises a nucleic acid sequence encoding a polypeptide comprising an amino acid sequence selected from the group consisting of:
a) A polypeptide comprising SEQ ID NO: 1;
b) A polypeptide comprising amino acids 23 to 449 of SEQ ID NO: 1;
c) A polypeptide comprising amino acids 35 to 449 of SEQ ID NO: 1;
d) A polypeptide comprising amino acids 23 to 227 of SEQ ID NO: 1;
e) A polypeptide comprising amino acids 35 to 227 of SEQ ID NO: 1;
f) A polypeptide comprising amino acids 228 to 449 of SEQ ID NO: 1;
g) A mutein of any of (a) to (f), wherein the amino acid sequence has at least 40% or 0% or 60% or 70% or 80% or 90% identity to at least one of the sequences in (a) to (e);
h) A mutein of any of (a) to (f) which is encoded by a DNA sequence which hybridizes to the complement of the native DNA sequence encoding any of (a) to (f) under moderately stringent conditions or under highly stringent conditions;
i) A mutein of any of (a) to (f) wherein any changes in the amino acid sequence are conservative amino acid substitutions to the amino acid sequences in (a) to (f); or an isoform, fused protein, functional derivative, active fraction or circularly permutated derivative of any of (a) to (f).

The nucleic acid may e.g. be administered as a naked nucleic acid molecule, e.g. by intramuscular injection.

It may further comprise vector sequences, such as viral sequence, useful for expression of the gene encoded by the nucleic acid molecule in the human body, preferably in the appropriate cells or tissues.

Therefore, in a preferred embodiment, the nucleic acid molecule further comprises an expression vector sequence. Expression vector sequences are well known in the art, they comprise further elements serving for expression of the gene of interest. They may comprise regulatory sequence, such as promoter and enhancer sequences, selection marker sequences, origins of multiplication, and the like. A gene therapeutic approach is thus used for treating and/or preventing the disease. Advantageously, the expression of clusterin will then be in situ.

In a preferred embodiment of the invention, the expression vector may be administered by intramuscular injection.

The use of a vector for inducing and/or enhancing the endogenous production of clusterin in a cell normally silent for expression of clusterin, or which expresses amounts of clusterin which are not sufficient are also contemplated according to the invention. The vector may comprise regulatory sequences functional in the cells desired to express clusterin. Such regulatory sequences may be promoters or enhancers, for example. The regulatory sequence may then be introduced into the appropriate locus of the genome by homologous recombination, thus operably linking the regulatory sequence with the gene, the expression of which is required to be induced or enhanced. The technology is usually referred to as “endogenous gene activation” (EGA), and it is described e.g. in WO 91/09955.

The invention further relates to the use of a cell that has been genetically modified to produce clusterin in the manufacture of a medicament for the treatment and/or prevention of peripheral neurological diseases.

The invention further relates to a cell that has been genetically modified to produce clusterin for manufacture of a medicament for the treatment and/or prevention of neurological diseases. Thus, a cell therapeutic approach may be used in order to deliver the drug to the appropriate parts of the human body.

The invention further relates to pharmaceutical compositions, particularly useful for prevention and/or treatment of peripheral neurological diseases, which comprise a therapeutically effective amount of clusterin and a therapeutically effective amount of an Heparin, optionally further a therapeutically effective amount of an immuno-suppressant.

The invention further relates to pharmaceutical compositions, particularly useful for prevention and/or treatment of peripheral neurological diseases, which comprise a therapeutically effective amount of clusterin and a therapeutically effective amount of an interferon, optionally further a therapeutically effective amount of an immuno-suppressant.

The invention further relates to pharmaceutical compositions, particularly useful for prevention and/or treatment of peripheral neurological diseases, which comprise a therapeutically effective amount of clusterin and a therapeutically effective amount of osteopontin, optionally further a therapeutically effective amount of an immuno-suppressant.

The definition of “pharmaceutically acceptable” is meant to encompass any carrier, which does not interfere with effectiveness of the biological activity of the active ingredient and that is not toxic to the host to which it is administered. For example, for parenteral administration, the active protein(s) may be formulated in a unit dosage form for injection in vehicles such as saline, dextrose solution, serum albumin and Ringer's solution.

The active ingredients of the pharmaceutical composition according to the invention can be administered to an individual in a variety of ways. The routes of administration include intradermal, transdermal (e.g. in slow release formulations), intramuscular, intraperitoneal intravenous, subcutaneous, oral, epidural, topical, intrathecal, rectal, and intranasal routes. Any other therapeutically efficacious route of administration can be used, for example absorption through epithelial or endothelial tissues or by gene therapy wherein a DNA molecule encoding the active agent is administered to the patient (e.g. via a vector), which causes the active agent to be expressed and secreted in vivo. In addition, the protein(s) according to the invention can be administered together with other components of biologically active agents such as pharmaceutically acceptable surfactants, excipients, carriers, diluents and vehicles.

For parenteral (e.g. intravenous, subcutaneous, intramuscular) administration, the active protein(s) can be formulated as a solution, suspension, emulsion or lyophilised powder in association with a pharmaceutically acceptable parenteral vehicle (e.g. water, saline, dextrose solution) and additives that maintain isotonicity (e.g. mannitol) or chemical stability (e.g. preservatives and buffers). The formulation is sterilized by commonly used techniques.

The bioavailability of the active protein(s) according to the invention can also be ameliorated by using conjugation procedures which increase the half-life of the molecule in the human body, for example linking the molecule to polyethylenglycol, as described in the PCT Patent Application WO 92/13095.

The therapeutically effective amounts of the active protein(s) will be a function of many variables, including the type of protein, the affinity of the protein, any residual cytotoxic activity exhibited by the antagonists, the route of administration, the clinical condition of the patient (including the desirability of maintaining a non-toxic level of endogenous clusterin activity).

A “therapeutically effective amount” is such that when administered, the clusterin exerts a beneficial effect on the peripheral neurological disease. The dosage administered, as single or multiple doses, to an individual will vary depending upon a variety of factors, including clusterin pharmacokinetic properties, the route of administration, patient conditions and characteristics (sex, age, body weight, health, size), extent of symptoms, concurrent treatments, frequency of treatment and the effect desired.

Clusterin can preferably be used in an amount of about 0.001 to 10 mg/kg or about 0.01 to 5 mg/kg or body weight or about 0.1 to 3 mg/kg of body weight or about 1 to 2 mg/kg of body weight. Further preferred amounts of clusterin are amounts of about 0.1 to 1000 μg/kg of body weight or about 1 to 100 μg/kg of body weight or about 10 to 50 μg/kg of body weight

The route of administration, which is preferred according to the invention, is administration by subcutaneous route. Intramuscular administration is further preferred according to the invention.

In further preferred embodiments, clusterin is administered daily or every other day.

The daily doses are usually given in divided doses or in sustained release form effective to obtain the desired results. Second or subsequent administrations can be performed at a dosage which is the same, less than or greater than the initial or previous dose administered to the individual. A second or subsequent administration can be administered during or prior to onset of the disease.

According to the invention, clusterin can be administered prophylactically or therapeutically to an individual prior to, simultaneously or sequentially with other therapeutic regimens or agents (e.g. multiple drug regimens), in a therapeutically effective amount, in particular with an interferon. Active agents that are administered simultaneously with other therapeutic agents can be administered in the same or different compositions.

The invention further relates to a method for treating a peripheral neurological disease comprising administering to a patient in need thereof an effective amount of clusterin, or of an agonist of clusterin activity, optionally together with a pharmaceutically acceptable carrier.

A method for treating a peripheral neurological disease comprising administering to a patient in need thereof an effective amount of clusterin, or of an agonist of clusterin activity, and heparin, optionally together with a pharmaceutically acceptable carrier.

A method for treating a peripheral neurological disease comprising administering to a patient in need thereof an effective amount of clusterin, or of an agonist of clusterin activity, and an interferon, optionally together with a pharmaceutically acceptable carrier, is also within the present invention.

A method for treating a peripheral neurological disease comprising administering to a patient in need thereof an effective amount of clusterin, or of an agonist of clusterin activity, and osteopontin, optionally together with a pharmaceutically acceptable carrier.

All references cited herein, including journal articles or abstracts, published or unpublished U.S. or foreign patent application, issued U.S. or foreign patents or any other references, are entirely incorporated by reference herein, including all data, tables, figures and text presented in the cited references. Additionally, the entire contents of the references cited within the references cited herein are also entirely incorporated by reference.

Reference to known method steps, conventional methods steps, known methods or conventional methods is not any way an admission that any aspect description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various application such specific embodiments, without undue experimentation, without departing from the general concept of the present invention. Therefore, such adaptations and modifications are intended to be within the meaning range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

Having now described the invention, it will be more readily understood by reference to the following examples that are provided by way of illustration and are not intended to be limiting of the present invention.

### EXAMPLES

### Example 1

### Recombinant Expression of Clusterin

Tagged recombinant murine or recombinant human clusterin (respectively mclusterin and hclusterin) was expressed in HEK cells and purified as follows:

The culture medium sample (100 ml) containing the recombinant protein with a C-terminal tag was diluted with one volume cold buffer A (50 mM NaH₂PO₄; 600 mM NaCl; 8.7% (w/v) glycerol, pH 7.5) to a final volume of 200 ml. The sample was filtered through a 0.22 um sterile filter (Millipore, 500 ml filter unit) and kept at 4° C. In a sterile square media bottle (Nalgene).

The purification was performed at 4° C. on the VISION workstation (Applied Biosystems) connected to an automatic sample loader (Labomatic). The purification procedure was composed of two sequential steps, affinity chromatography specific for the tag followed by gel filtration on a Sephadex G-25 medium (Amersham Pharmacia) column (1.0×10 cm).

The first chromatography step resulted in the eluted protein collected in a 1.6 ml fraction.

For the second chromatography step, the Sephadex G-25 gel-filtration column was regenerated with 2 ml of buffer D (1.137 M NaCl: 2.7 mM KCl; 1.5 mM KH₂PO₄; 8 mM Na₂HPO₄; pH 7.2), and subsequently equilibrated with 4 column volumes of buffer C (137 mM NaCl; 2.7 mM KCl; 1.5 mM KH₂PO₄; 8 mM Na₂HPO₄; 20% (w/v) glycerol; pH 7.4). The peak fraction eluted from the forst step affinity column was automatically through the integrated sample loader on the VISION loaded onto the Sephadex G-25 column and the protein was eluted with buffer C at a flow rate of 2 ml/min. The desalted sample was recovered in a 2.2 ml fraction. The fraction was filtered through a 0.22 um sterile centrifugation filter (Millipore), frozen and stored at −80° C. An aliquot of the sample was analyzed on SDS-PAGE (4-12% NuPAGE gel; Novex) by coomassie staining and Western blot with anti-tag antibodies.

Coomassie staining. The NuPAGE gel was stained in a 0.1% coomassie blue R250 staining solution (30% methanol, 10% acetic acid) at room temperature for 1 h and subsequently destained in 20% methanol, 7.5% acetic acid until the background was clear and the protein bands clearly visible.

Western blot. Following the electrophoresis the proteins were electrotransferred from the gel to a nitrocellulose membrane at 290 mA for 1 hour at 4° C. The membrane was blocked with 5% milk powder in buffer E (137 mM NaCl; 2.7 mM KCl; 1.5 mM KH₂PO₄; 8 mM Na₂HPO₄; 0.1% Tween 20, pH 7.4) for 1 h at room temperature, and subsequently incubated with a mixture of 2 rabbit polyclonal anti-tag antibodies (G-18 and H-15, 0.2 ug/ml each; Santa Cruz) in 2.5% milk powder in buffer E overnight at 4° C. After further 1 hour incubation at room temperature, the membrane was washed with buffer E (3×10 min), and then incubated with a secondary HRP-conjugated anti-rabbit antibody (DAKO, HRP 0399) diluted 1/3000 in buffer E containing 2.5% milk powder for 2 hours at room temperature. After washing with buffer E (3×10 minutes), the membrane was developed with the ECL kit (Amersham Pharmacia) for 1 min. The membrane was subsequently exposed to a Hyperfilm (Amersham Pharmacia), the film developed and the western blot image visually analysed.

Protein assay. The protein concentration was determined using the BCA protein assay kit (Pierce) with bovine serum albumin as standard. The average protein recovery was 216 μg purified clusterin per 100 ml culture medium.

Analysis of the purified protein in non-reducing SDS PAGE showed that the recombinant protein had the heterodimeric structure of native clusterin (not shown).

### Example 2

### Protective Effect of Clusterin on Neuropathy Induced by Sciatic Nerve Crush in Mice

Abbreviations

CMAP: compound muscle action potential

DAC: day after crush

DIV: days in vitro

EMG: electromyography

IGF-1: insulin-like growth factor

i.p.: intraperitoneal

i.v.: intravenous

s.c.: subcutaneous

s.e.m.: standard error of the mean

vs: versus

Introduction

The present study was carried out to evaluate nerve regeneration in mice treated with clusterin at different doses. In this model a positive effect of clusterin on neuronal and axonal (sensory and motor neurons) survival and regeneration, on myelination or macrophage inflammation could lead to a restoration of motor function. The regeneration may be measured according to the restoration of sensorimotor functions and morphological studies. Therefore in the present work electrophysiological recordings and histomorphometric analysis were performed in parallel.

Materials and Methods

Animals

Seventy-two 8 weeks-old females C57bl/6 RJ mice (Elevage Janvier, Le Genest-St-Isle, France) were used. They were divided into 6 groups (n=12): (a) vehicle sham operated group; (b) vehicle nerve crush operated group; (c) nerve crush/mclusterin (300 μg/kg); (d) nerve crush/mclusterin (1000 μg/kg); (e) nerve crush/4-methylcatechol (10 μg/kg); (f) nerve crush/osteopontin (100 μg/kg). Osteopontin (OPN) is a highly phosphorylated sialoprotein that is a prominent component of the mineralized extracellular matrices of bones and teeth. Its use or the use of or of an agonist of its activity, is claimed in WO02092122 for the manufacture of a medicament for the treatment and/or prevention of a neurologic disease.

They were group-housed (12 animals per cage) and maintained in a room with controlled temperature (21-22° C.) and a reversed light-dark cycle (12 h/12 h) with food and water available ad libitum. All experiments were carried out in accordance with institutional guidelines.

Lesion of the Sciatic Nerve

The animals were anaesthetized with i.p. injection of 60 mg/kg ketamine chlorhydrate (Imalgéne 500®, Rhòne Médreux, Lyon, France). The right sciatic nerve was surgically exposed at mid thigh level and crushed at 5 mm proximal to the trifurcation of the sciatic nerve. The nerve was crushed twice for 30 s with a haemostatic forceps (width 1.5 mm, Koenig; Strasbourg, France) with a 90 degree rotation between each crush.

Planning of Experiments and Pharmacological Treatment

Electromyographical (EMG) testing was performed once before the surgery day (baseline) and each week during 2 weeks following the operation.

The day of nerve crush surgery was considered as day (D) 0. No test was performed during the 4 days following the crush.

Body weight and survival rate were recorded every day.

From the day of nerve injury to the end of the study, mclusterin (recombinant mclusterin from HEK cell) or 4-methylcatechol was administered daily by intraperitoneal (i.p) route, whereas daily injection of osteopontin was performed subcutaneous (s.c.).

At the 2nd week, 4 animals per group were sacrificed and sciatic nerve was dissected to perform morphological analysis.

Electrophysiological Recording

Electrophysiological recordings were performed using a Neuromatic 2000M electromyograph (EMG) (Dantec, Les Ulis, France). Mice were anaesthetized by intraperitoneal injection of 100 mg/kg ketamine chlorhydrate (Imalgene 500®, Rhòne Mérieux, Lyon, France). The normal body temperature was maintained at 30° C. with a heating lamp and controlled by a contact thermometer (Quick, Bioblock Scientific, Iilkirch, France) placed on the tail.

Compound muscle action potential (CMAP) was measured in the gastrocnemius muscle after a single 0.2 ms stimulation of the sciatic nerve at a supramaximal intensity (12.8 mA). The amplitude (mV), the latency (ms) and the duration (time needed for a depolarization a nd a repolarization session) of the action potential were measured. The amplitude is indicative of the number of active motor units, while the distal latency indirectly reflects motor nerve conduction and neuromuscular transmission velocities.

Morphometric Analysis

Morphometric analysis was performed 2 weeks after the nerve crush. Four randomly selected animals per groups were used for this analysis. Mice were anesthetized with i.p. injection of 100 mg/kg imalgene 500®. A 5 mm segment of sciatic nerve was excised for histology. The tissue was fixed overnight with a 4% aqueous solution glutaraldehyde (Sigma, L'isle d'Abeau-Chesnes, France) in phosphate buffer solution (pH=7.4) and maintained in 30% sucrose at 4° C. until use. The nerve was fixed in 2% osmium tetroxide (Sigma, L'Isle d'Abeau-Chesnes, France) in phosphate buffer for 2 hr and dehydrated in serial alcohol solutions and embedded in Epon. Embedded tissues were then placed at 70° C. during 3 days for polymerisation. Transverse sections of 1.5 μm were made with a microtome and stained of 1% of toluidine blue (Sigma, L'isle d'Abeau-Chesnes, France) for 2 min and dehydrated and mounted in Eukitt. Cross sections were obtained at the middle of the crush site. Morphometric analysis and fiber counts were performed on the total area of the nerve section using a semi-automated digital image analysis software (Biocom, France). The proportions of degenerating and non -degenerating myelinated fibers were analysed. Myelinated fibers showing multi-lobular axoplasm and/or irregular myelin sheath were considered as fibers undergoing processes of degeneration. The following parameters were calculated: axon area, myelin area and fiber area (axon and myelin area).

Data Analysis

Global analysis of the data was performed using one factor or repeated measure analysis of variance (ANOVA) and one-way ANOVA, and non-parametric tests (Mann Whitney test). Dunnett's test was used further when appropriate. The level of significance was set at p<0.05. The results were expressed as mean±standard error of the mean (s.e.m.).

Results

All animals survived after the nerve crush procedures. Throughout the study, several mice died: on day 2, mouse n° 8 from the nerve crushlosteopontin group and nerve mouse n° 12 from the crush/mclusterin at 1 mg/kg group: on day 7 mouse n° 9 from the nerve crush/vehicle group and n° 9 from the nerve crush/mclusterin at 1 mg/kg group, due to the anesthetic.

Animal Weight

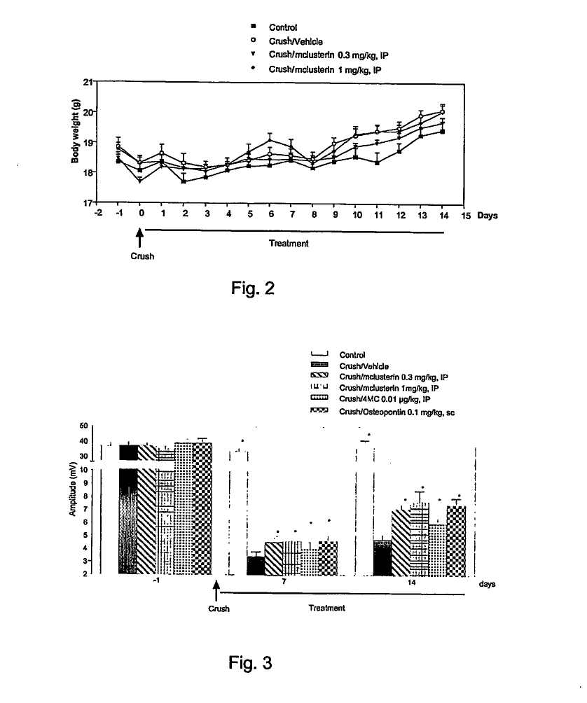

As illustrated in FIG. 2, all animals showed slight decrease in their body weight during 2-3 days following the surgery. Then, animals showed a progressive recovery of their body weight. The different treatments with mclusterin, did not induce any significant changes in the body weight of mice with crushed sciatic nerve when compared to untreated mice.

Electrophysiological Measurements

Amplitude of the Compound Muscular Action Potential (FIG. 3):

In sham-operated animals, there was not significant change in the CMAP amplitude throughout the study. In contrast, crush of the sciatic nerve induced a dramatic decrease in the amplitude of CMAP with a decrease >90% at D7 and D14 when compared to the respective levels of sham-operated animals. When mice with crushed sciatic nerve were treated with clusterin, at 300 μg/kg or 1 mg/kg, or osteopontin at 100 μg/kg, they demonstrated a sgnificant increase (about 1.5 times) in the CMAP amplitude as compared to the level in untreated mice. Similarly, 4-MC treatment also enhanced the CMAP amplitude of mice with nerve crush, but to a lesser extent than clusterin or osteopontin.

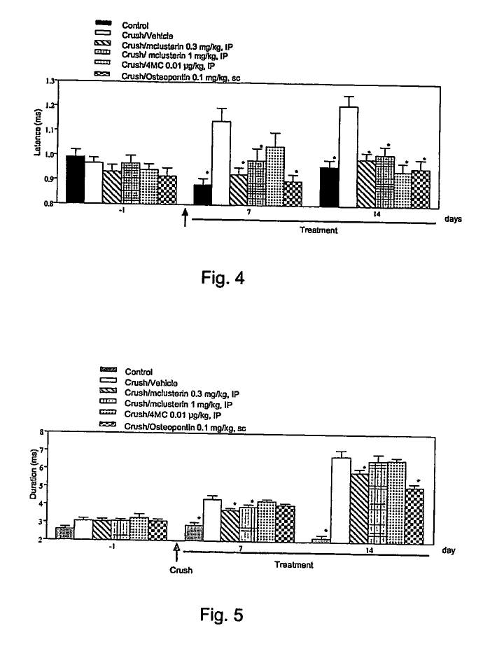

Latency of the Compound Muscular Action Potential (FIG. 4):

In sham-operated animals, there was no deterioration of CMAP latency throughout the study. In contrast, mice with crushed sciatic nerve showed 1.2 times greater CMAP latency than sham-operated animals. In mice with crushed sciatic nerve treated with clusterin or osteopontin, the CMAP latency value was significantly reduced as compared to the one of untreated mice. At day 7, this effect could be observed after treatment with 0.3 mg/kg of clusterin and 0.1 mg/kg of osteopontin. At day 14, both concentrations of clusterin were efficacious.

Duration of the Compound Muscular Action Potential (FIG. 5):

In sham-operated animals, the duration of CMAP was not statistically different to the baseline value. In contrast, mice with crushed sciatic nerve showed a significant extension of CMAP duration, especially at D14 where the duration was 3 times greater than in sham-operated animals.

When mice with crushed sciatic nerve were treated with clusterin at 300 μg/kg or osteopontin, they demonstrated a significantly reduced CMAP duration as compared to the vehicle treated animals with nerve crush.

Morphometric Analysis

The morphometric analysis was carried out after termination of the experiment at day 14.

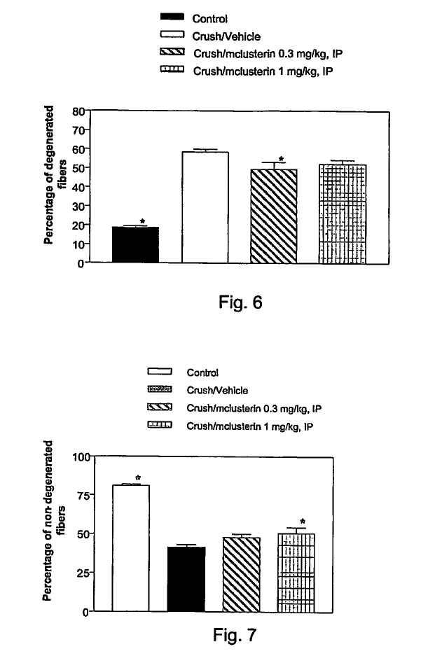

Percentage of Degenerated (FIG. 6) and Non-Degenerated Fibers (FIG. 7)

As shown in FIG. 6, the percentage of degenerated fibers in sciatic nerve of sham-operated animals (control) was <20%. When the sciatic nerve was subjected to a crush, the proportion of degenerated fibers was significantly increased up to 60% (crush/vehicle). Treatment of mice with 300 μg/kg or 1 mg/kg of clusterin induced a significant decrease in the proportion of degenerated fibers as compared to the untreated group.

Conversely, the proportion of non-degenerated fibers in sham-operated animals (control) was two times greater than in untreated mice with crushed sciatic nerve (crush/vehicle) (FIG. 7). Treatment with clusterin at 300 μg/kg or 1 mg/kg induced a significant increase in the density of non-degenerated fibers.

Conclusions

The nerve-crush model is a very dramatic model of peripheral neuropathy. Immediately after the nerve crush most of the fibers having a big diameter are lost, due to the mechanical injury, leading to the strong decrease in the CMAP amplitude. The CMAP latency is not immediately affected but shows an increase at 14 days due to additional degeneration of small diameter fibers by secondary, immune mediated degeneration (macrophages, granulocytes). The CMAP duration is increased at day 7 and peaks at day 14. At 21 days (not shown), crush lesions allow for regeneration, an additional process of interest in relation to neuropathic states.

Clusterin showed a protective effect in the nerve crush model in mice on all parameters measured. Morphological studies performed 2 weeks post crush show a significant decrease in the percentage of degenerating fibers and an increase in total fiber number. Clusterin is as effective as the control molecule used in this study, 4-methylcatechol. This positive effect on functional and histological recovery may be due to clusterin effects on:
direct protection of fibers from secondary immune mediated degeneration;
accelerated remyelination and protection of axons;
accelerated regeneration/sprouting of damaged axons;
increased myelin debris clean up by macrophages.
modulation of macrophage response to axotomy.

### Example 3

### Subcutaneous Administration of Clusterin Accelerates Functional Recovery After Sciatic Nerve Crush

Introduction

To study the long lasting effect of clusterin treatment on nerve regeneration, a second group of mice was treated for four weeks by daily (5 times/week, s.c.) administration of recombinant human clusterin produced in HEK cells.

Materials and Methods

Mice were divided into 6 groups (n=6) as follows:
(a) vehicle nerve crush operated group;
(b) nerve crush/h-IL6 (30 μg/kg);
(c) nerve crush/hclusterin (0.1 mg/kg);
(d) nerve crush/hclusterin (300 μg/kg);
(e) nerve crush/hclusterin (1 mg/kg).

The procedures described under Example 2 were performed, except that animals received a subcutaneous injection (100 μl/mouse) of recombinant recombinant human clusterin produced in HEK cells (hclusterin) instead of intra-peritoneal injection of recombinant mouse clusterin. The vehicle was NaCl 0.9%, BSA 0.02%. The positive control was recombinant human IL-6 (30 μg/kg, s.c.). Electromyographic and body weight parameters were evaluated as previously described.

Electrophysiological Recording

The compound muscle action potential (CMAP) was measured in the gastrocnemius muscle after a single 0.2 ms stimulation of the sciatic nerve at a supramaximal intensity (12.8 mA). Various parameters i.e. the amplitude (mV), the latency (ms) and the duration of the action potential were evaluated as previously described at 0, 7, 14, 21 and 28 days after crush on the gastrocnemius muscle of the crushed side (ipsilateral) and on the gastrocnemius muscle of the opposite side (contralateral).

Choline Acetyl Transferase (ChAT) Activity

After the four weeks of treatment, described in example 3, mice were anesthetized and sacrificed. The contralateral and ipsilateral gastrocnemius muscles were collected and analyzed for choline acetyl transferase (ChAT) activity, a indicator of neuronal innervation. The ChAT activity was measured accordingly to the protocol described by Contreras et al. (Contreras et al., 1995) except that cold acetyl-CoA was omitted and 0.25 nmol of ³H-acetyl-CoA corresponding to 0.05 μCi were added.

Neurofilaments-High Molecular Weight Form (NF-H)

NF-H and its phosphorylated forms are indicators of axonal maturation (Riederer et al., 1996). After the four weeks of treatment, described in example 3, mice were anesthetized and sacrificed. Nerves were collected and extracted in triple detergent buffer and samples were processed for protein content by a protein assay kit (Pierce) and for NF-H quantification by sandwich ELISA.

For the NF-H ELISA, the protocol used was the following: the capture antibody, mouse monoclonal antibody SMI 31 (anti-NF-H phosphorylated 1/2500; Sternberger), was incubated in PBS overnight at 4° C. The plates were blocked with PBS containing 1% BSA for 1 hours. After incubation for 2 hours with the samples, the detection antibody, rabbit polyclonal N4142 anti-NF (1/1000; Sigma), was diluted in PBS-BSA, incubated for 2 hours and revealed by peroxidase after incubation with anti-rabbit HRP conjugated antibody (1/3000, Sigma: diluted in PBS-BSA, 1 hours). Each optic density read at 492 nm was reported to a standard curve of bovine NF-H (Sigma) and then to the content of protein of each sample.

Results

Electrophysiological Measurements

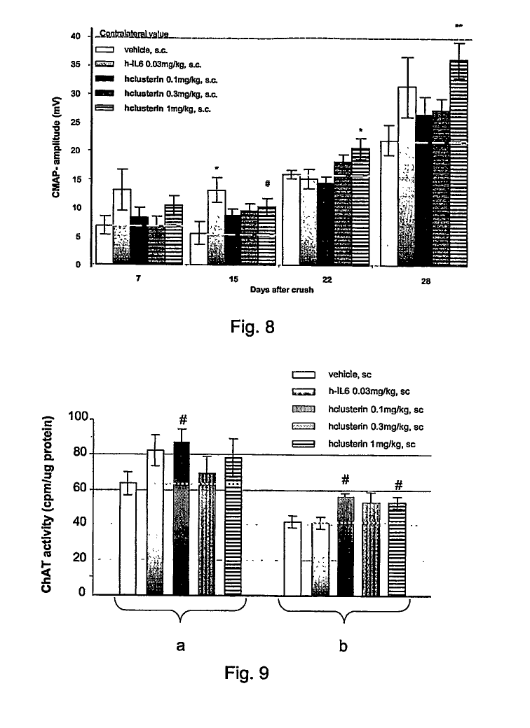

Amplitude of the Compound Muscular Action Potential (FIG. 8):

One week after crush, the CMAP amplitude was not significantly different between animals treated with IL-6 (30 μg/kg), hclusterin (100, 300 or 1000 μg/kg) or vehicle treated group. From day 15 to day 28, mice with crushed sciatic nerve treated with hclusterin and IL-6 demonstrated a progressive increase of the CMAP amplitude. After 4 weeks, the CMAP amplitude of mice treated with clusterin, as compared to the level in untreated mice, showed a very significant increase.

Latency of the Compound Muscular Action Potential:

The latency of the compound muscle action potential was measured in neuropathic mice treated with vehicle, recombinant human IL-6 (30 μg/kg) or hclusterin (100, 300 and 1000 ,ug/kg). Ipsilateral and contralateral measures were taken at 1, 2, 3, or 4 weeks after sciatic nerve injury. The results are reported in the following table (Table 1):

TABLE 1

| | Latency (ms) | | | | | Latency (ms) | | | |
|---|---|---|---|---|---|---|---|---|---|
| | Contralateral | Contralateral | Contralateral | Contralateral | | Ipsilateral | Ipsilateral | Ipsilateral | Ipsilateral |
| | 7 DAC | 14 DAC | 21 DAC | 28 DAC | | 7 DAC | 14 DAC | 21 DAC | 28 DAC |
| Vehicle | 0.79 | 0.85 | 0.87 | 0.79 | Vehicle | 0.97# ^{a} | 0.97 | 1.32*** ^{a} | 1.08** ^{a} |
| | 0 04 | 0 03 | 0 04 | 0 06 | | 0.09# | 0.07 | 0.09*** | 0.06** |
| h-IL6 | 0.77 | 0.81 | 0.74 | 0.77 | h-IL6 | 0.95 | 0.91 | 0.91* ^{b} | 0.91** ^{b} |
| 30 μg/kg | 0 04 | 0 05 | 0 03 | 0 05 | 30 μg/kg | 0 09 | 0 04 | 0.10 | 0.04 |
| clusterin | 0.80* ^{b} | 0.79 | 0.74 | 0.69 | clusterin | 0.93 | 0.89 | 1.04* ^{b} | 0.94 |
| 0 1 mg/kg | 0.02 | 0 05 | 0 06 | 0 01 | 0.1 mg/kg | 0 06 | 0 01 | 0.08 | 0 07 |
| clusterin | 0.83 | 0.88 | 0.87 | 0.70 | clusterin | 0.92 | 0.90 | 0.89** ^{b} | 0.90# ^{b} |
| 0 3 mg/kg | 0.04 | 0 02 | 0.02 | 0 04 | 0.3 mg/kg | 0 09 | 0.04 | 0.05 | 0.06 |
| clusterin | 0.85 | 0.86 | 0.79 | 0.76 | clusterin | 0.83 | 0.91 | 1.04* ^{b} | 0.97 ^{b} |
| 1 mg/kg | 0.04 | 0 03 | 0.03 | 0 05 | 1 mg/kg | 0 04 | 0.03 | 0.06 | 0 06 |

^{a} Anova single factor test against contralateral values
^{b} Anova single factor test against vehicle treated group
The numbers in italic represent the standard errors (SD)
N = 6 mice/group;
#p < 0.1,
*p < 0.05,
**p < 0.01,
***p <0.005

There was no deterioration of the CMAP latency on the contralateral side throughout the study with an exception at 7 DAC for mice treated with 0.1 mg/kg of clusterin. In contrast, on the ipsilateral side CMAP latency increased after the crush. In mice treated with IL-6 and clusterin, the ipsilateral CMAP latency was significantly reduced as compared to the one of untreated mice. At 21 and 28 DAC, recombinant hIL-6 and clusterin administration (1 and 0.3 mg/kg) significantly improved latency recovery.

Duration of the Compound Muscular Action Potential

As for the latency above, the duration of the compound muscular action was measured for all groups on the contralateral and ipsilateral sides and the results reported in Table 2 below.

TABLE 2

| | Duration (ms) | | | | | Duration (ms) | | | |
|---|---|---|---|---|---|---|---|---|---|
| | Contralateral | Contralateral | Contralateral | Contralateral | | Ipsilateral | Ipsilateral | Ipsilateral | Ipsilateral |
| | 7 DAC | 14 DAC | 21 DAC | 28 DAC | | 7 DAC | 14 DAC | 21 DAC | 28 DAC |
| Vehicle | 2.0 | 2.5 | 2.9 | 2.9 | Vehicle | 3.5# ^{a} | 4.4** ^{a} | 3.8 | 3.0 |
| | 0.1 | 0 2 | 0.2 | 0 2 | | 0.7 | 0.4 | 0 8 | 0.2 |
| h-IL6 | 2.5# | 2.0* ^{b} | 3.0 | 2.9 | h-IL6 | 2.6 | 3.6 | 3.4 | 3.0 |
| 30 μg/kg | 0.3 | 0.2 | 0 2 | 0 1 | 30 μg/kg | 0 2 | 0 3 | 0 1 | 0 1 |
| clusterin | 2.6* ^{b} | 2.0* ^{b} | 2.9 | 2.9 | clusterin | 2.7 | 4.1 | 3.2 | 2.9 |
| 0 1 mg/kg | 0.2 | 0.1 | 0 3 | 0 1 | 0 1 mg/kg | 0 1 | 0 5 | 0 2 | 0 3 |
| clusterin | 2.3* ^{b} | 2.2 | 2.9 | 3.0 | clusterin | 2.7 | 3.9 | 2.0*** ^{b} | 3.4 |
| 0 3 mg/kg | 0.1 | 0 2 | 0 2 | 0 1 | 0 3 mg/kg | 0 1 | 0 4 | 0.1 | 0 2 |
| clusterin | 2.1 | 2.1 | 2.6 | 2.9 | clusterin | 2.6 | 3.4# ^{b} | 3.0# ^{b} | 3.1 |
| 1 mg/kg | 0 1 | 0 2 | 0 3 | 0 1 | 1 mg/kg | 0.2 | 0.4 | 0.2 | 0.2 |

^{a} Anova single factor test against contralateral values
^{b} Anova single factor test against vehicle treated group
The numbers in italic represent the standard errors (SD)
N = 6 mice/group;
#p < 0.1,
*p < 0.05,
**p < 0.01,
***p <0.005

In vehicle treated group, the duration of the ipsilateral CMAP increased after crush and returned to the contralateral value after 4 weeks. Clusterin treatments (1 and 0.3 mg/kg) diminished the overall increase of CMAP duration and accelerated the recovery.

Choline Acetyl Transferase (ChAT) Activity (FIG. 9):

Four weeks after crush, the ChAT activity in the ipsilateral gastrocnemius muscle (FIG. 9.a) was not fully restored. Clusterin treatment slightly favored (p<0.1) the recovery of ChAT activity on gastrocnemius muscle. The ChAT content in the contralateral muscle of mice treated with hClusterin showed an increase as compared to vehicle treated animals (FIG. 9.b).

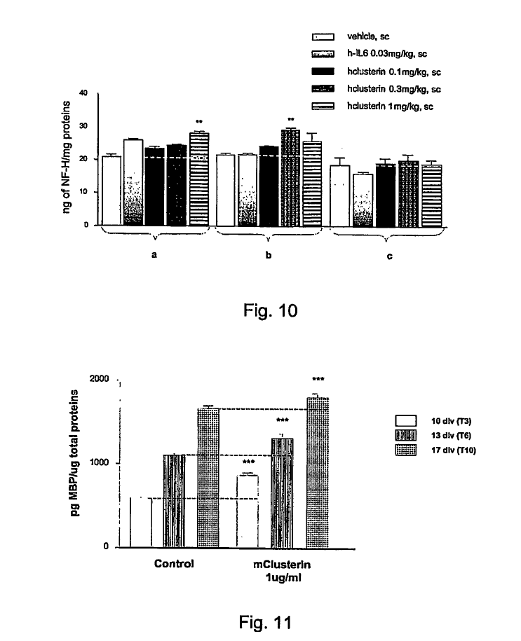

Neurofilaments-High Molecular Weight Form (NF-H) (FIG. 10):

Four weeks after the crush, in the vehicle treated group, the levels of NF-H in the proximal part of sciatic nerve (above the crush site; FIG. 10.b) and in the distal part (below the crush site; FIG. 10.c) were not different as compared to the level of NF-H in the contralateral nerve (FIG. 10.a). Clusterin treatment increased the content of NF-H on the contralateral side and on the proximal part of the crushed nerve.

Conclusion

These results as those obtained after 15 days of treatment (Example 2), highlighted the beneficial effect of clusterin in treating nerve-crush model. Depending of the time of treatment, the effect could be seen on all studied parameters of compound action muscle potential (CAMP) namely the latency, the duration and the amplitude. Clusterin treatment also increased the ChAT and NF-H contents in crushed and contralateral nerves. No adverse effect was observed on body weight evolution (data not shown).

### Example 4

### Clusterin Stimulates Myelin Basic Protein (MBP) Formation in Maturating Hippocampal Slice Cultures

Introduction

Regulation of nerve regeneration after injury or disease requires not only axonal sprouting and elongation but also new myelin synthesis. Myelination is necessary for the normal nerve conduction and axonal protection against excitotoxicity or immunologic attacks for examples. Because myelin repair is mostly a recapitulation of ontogenetic events (Capello et al., 1997; Kuhn et al., 1993), the organotypic hippocampal slices cultures were used to mimic developmental myelination. More precisely the myelin basic protein (MBP) level, a protein representative of matured oligodendrocytes and Schwann cells, was monitored by ELISA.

Materials and Methods

Organotypic Hippocampal Slice Cultures

Organotypic hippocampal slice cultures were prepared according to the method of Stoppini et al. (Stoppini et al., 1991). Briefly, hippocampi were obtained from five day-old C57/BI6 mice. Using a Mcillvain tissue chopper, 500-micron thick slices were cut. Slices were then disposed onto Millicell-CM inserts placed in 6 wells plates containing 1 ml of cultures medium (50% MEM, 25% HBSS, 25% horse serum). Cultures were maintained in 5% CO2 at 37° C. during the 6th days and then transferred at 33° C. Medium was changed every 3 days.

Developmental Myelination

Capacity of the clusterin to increase myelination that normally occurs during the first 3 weeks in vitro was tested.

Slices were first treated from day 7 until day 17 with mclusterin (1 μg/ml, 100 ng/ml and 10 ng/ml) in medium containing horse serum (25%). The treatments were renewed every 2 days.

At the end of treatment (i.e. 3, 6 and 10 days of treatment corresponding to 10, 13 and 17 days in vitro, respectively) slices (6 slices per group) were lysed in triple detergent buffer and MBP content were analyzed by MBP ELISA.

This experiment was performed twice and the results shown in FIG. 11.

Similar results were obtained when these experiments were reproduced with recombinant human clusterin produced in HEK or CHO cells instead of recombinant mouse clusterin and (data not shown).

MBP ELISA

After lyses at different time points, samples were processed for protein content by a protein assay kit (Pierce) and for MBP quantification by sandwich ELISA.

The protocol for the MBP-ELISA was the following. The capture antibody, mouse monoclonal antibody anti-MBP (1/5000; Chemicon), was diluted in PBS and incubated overnight at 4° C. The plates were blocked with PBS containing 1% BSA for 1 hours. Samples, diluted in PBS, were incubated for 2 hours. The detection antibody, rabbit polyclonal anti-MBP (1/300; Zymed) diluted in PBS-BSA, was incubated for 2 hours and revealed by peroxidase after incubation with anti-rabbit HRP conjugated antibody (1/3000, Sigma; diluted in PBS-BSA, 1 hours). Each optic density read at 492 nm was reported to a standard curve of MBP (InVitrogen) and then to the content of protein of each sample.

Results

At the starting culture time, hippocampal slices of P4 mice (4 days post-natal) were not expressing detectable level of MBP. As the hippocampal slices matured, the level of MBP detected by ELISA increased to reach a stable level after 21 days in vitro (DIV, data not shown).

Adding 10, 100 and 1000 ng/ml of recombinant hclusterin to the culture medium at 7, 10 or 14 DIV increased the MBP content of hippocampal slices cultures as assessed by MBP-ELISA performed three days after protein addition. The MBP content of slices treated with 1 μg/ml of mclusterin is shown in FIG. 11. This MBP increase is no more visible at 21 DIV when myelin development is finished (data not shown).

Similar results are obtained with the other concentrations of mclusterin (10 and 100 ng/ml) and with hclusterin (data not shown).

Conclusion

Clusterin stimulates MBP formation in hippocampal slice cultures without affecting the total amount detected in matured hippocampal slices.

### Example 5

### Clusterin Protects Against Demyelination of Hippocampal Slices by Anti-MOG Antibody with Baby Hamster Complement

Introduction

Breakdown of myelin, a characteristic of chronic inflammatory demyelinating polyneuropathy (CIDP) and Guillain-Barré syndrome (GBS), is thought to be due to the presence of autoimmune reaction against nerves, including myelin components (Ho et al., 1998; Kwa et al., 2003; Steck et al., 1998). In order, to mimic antibody-induced demyelination, an in vitro system was setup where organotypic hippocampal slice cultures were treated for two days by anti-MOG (myelin oligodendrocyte glycoprotein) antibody in combination with baby hamster complement. The treatment results in a specific demyelination since isotype matching control immunoglobulin treatment did not induce significant demyelination. This system was used to test the protective effect of clusterin in this paradigm, clusterin was added one day before and concomitantly with the demyelinating treatment and the MBP level was monitored by ELISA (see Example 4 for details).

Materials and Methods

Demyelinating Protocol

Slices, prepared as described in Example 4 (Organotypic hippocampal slice cultures section), were treated at the end of developmental myelination that occurs after 21 days in vitro (DIV).

Demyelination was induced by treating slices with anti-MOG antibodies associated with baby rabbit complement (1/60-1/30 depending of the batch; CL-3441, Cedarlane) during 2 days in 25% horse serum containing medium.

As controls, slices were treated with IgG1 not relevant antibodies (60 ug/ml; M-7894, Sigma) and complement or slices were untreated.

At the end of the treatment, slices (5 slices per group) were lysed in triple detergent buffer and myelin level content analyzed by MBP ELISA.

1 ug/ml, 100 ng/ml or 10 ng/ml of recombinant mouse clusterin were applied during 24 hours before demyelination treatment and add ed at the time of treatment (a total of 3 days).

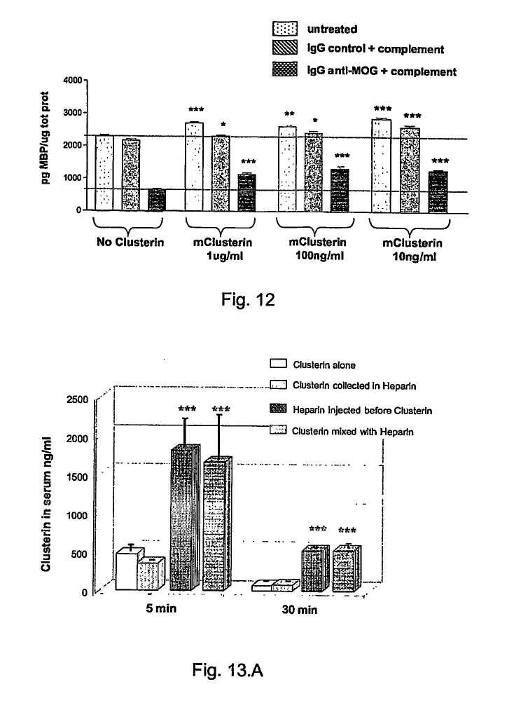

This experiment was performed three times and the results shown in FIG. 12.

Similar results were obtained when these experiments were reproduced with recombinant human clusterin produced in HEK or CHO cells instead of recombinant mouse clusterin and (data not shown).

Results

The results of this experiment are shown in FIG. 12. Adding as low as 10 ng/ml of clusterin to the medium at the time of anti-MOG/complement treatment significantly protected against demyelination.

Conclusion

In an autoimmune mediated demyelination model, clusterin protects against demyelination induced by anti-MOG and complement.

### Example 6

### Co-Injection of Clusterin with Heparin

Introduction

In serum, clusterin is known to bind several proteins (reviewed in Trougakos and Gonos (Trougakos and Gonos, 2002) and Jones and Jomary (Jones and Jomary, 2002) and presents several putative binding sites (see FIG. 1, scheme based on Rosenberg and Silkensen, 1995). Among them four are thought to be heparin-binding domains. In order to study the relevance of these heparin-binding domains on the bioavailability of clusterin, the effect of Heparin, in this case Liquemine (Roche), was tested on clusterin pharmacokinetiks.

Materials and Methods

First Experiment

Three groups (3 mice/group) of 8 weeks-old C57BI6 20 grams females were injected i.v. as follows:
Group 1: heparin (7500 U/kg) in 100 μl of NaCl 0.9%, 5 minutes before the injection of hclusterin (300 μg/kg) in 100 μl of NaCl 0.9%.
Group 2: mixed solution of hclusterin (300 μg/kg) and heparin (7500 U/kg) in 100 μl of NaCl 0.9%.
Group 3: 300 μg/kg of hclusterin alone.

Blood was collected at 5 and 30 minutes after clusterin injection into a tube. The blood of mice belonging to group 3 was collected in a tube either with or without heparin (+/− heparin. Then the presence of clusterin was studied in the ELISA test described below.

Second Experiment

Three groups (4 mice/group) of 8 weeks-old C57BI6 20 grams females were injected iv. as follows:
Group 1: heparin (7500 U/kg) in 100 μl of NaCl 0.9%, 5 minutes before the injection of hclusterin (1 mg/kg) in 100 μl of NaCl 0.9%. Group 1 received a mixed solution of clusterin (1 mg/kg) +heparin (7500 U/kg) in 100 μl of NaCl 0.9%.
Group 2: 1 mg/kg of hclusterin alone. Heparin (7500 U/kg) was injected 28 min after hclusterin injection (2 minutes before the 30 minutes bleeding point).
Group 3: 1 mg/kg of hclusterin alone

Blood was collected at 5 and 30 minutes after clusterin injection. Then clusterin level in serum was monitored using the ELISA test described below.

Clusterin ELISA

The sandwich ELISA was developed using monoclonal antibodies 41D (1/1000-50 μl, Upstate N. 05-354) as capture antibody. The residual binding sites were blocked at RT in Blocking Buffer (1%BSA (fraction V)/0.1% Tween-20 in 0.5M NaCl). Serum samples containing recombinant human clusterin were tested in serial dilutions in PBS. Followed by four washes in PBS/0.05% Tween-20. A tag Biotin conjugate (1/1000, Qiagen N.34440) was used as revealing antibody. The presence of revealing antibodies was monitored by Streptavidin-HRP (115000 in PBS, DAKO P0397) 1 hour at RT, followed by OPD reaction (Sigma).

Results

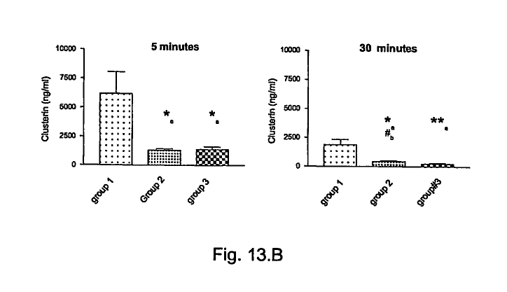

As shown in FIG. 13A, pre-incubation of clusterin with heparin (Clusterin mixed with Heparin) or pre-injection of heparin before clusterin injection (Heparin injected before Clusterin) greatly improved (p<0.005) the clusterin bio-availability in contrast, collection of clusterin in a heparin containing tube did not change the level of clusterin detected

When heparin was administered prior to the second bleeding (group 2 of the second experiment FIG. 13B), the clusterin level detected in the serum was significantly lower (p<0.05) than when heparin was co-injected with clusterin. Nevertheless, heparin injected prior to blood collection slightly increased the level of detectable clusterin as compared to clusterin alone (p<0.1).

Conclusions

Heparin administration significantly improved clusterin bio-availability (FIG. 13A). However to be fully efficient the heparin has to be injected before or concomitantly to clusterin delivery (FIG. 13B).

### REFERENCES

1. Altschul, S. F., Gish, W., Miller, W., Myers, E. W., and Upman, D. J. (1990). Basic local alignment search tool. J. Mol. Biol. 215, 403-410.

2. Alshul, S. F., Madden, T. L., Schaffer, A. A., Zhang, J., Zhang, Z, Miller, W., and Lipman, D. J. (1997). Gapped BLAST and PSI-BLAST: a new generation of protein database search programs. Nucleic Acids Res. 25 3389-3402.

3. Bonnard, A. S., Chan, P., and Fontaine, M. (1997). Expression of clusterin and C4 mRNA during rat peripheral nerve regeneration . Immunopharmacology 38, 81-86.

4. Capello, E., Voskuhl, R. R., McFarland, H. F., and Raine, C. S. (1997). Multiple sclerosis: re-expression of a developmental gene in chronic lesions correlates with remyelination. Ann. Neurol. 41, 797-805.

5. Contreras, P. C., Steler, C., Yu, E., Callison, K., Stong, D., and Vaught, J. L. (1995). Systemic administration of rhIGF-1 enhanced regeneration after sciatic nerve crush in mice. J Pharmacol Exp Ther. 274, 1443-1449.

6. Derynck, R., Content, J., DeClercq, E., Volckaert, G., Tavemler, J., Devos, R., and Flers, W. (1980), isolation and structure of a human fibroblast interferon gene. Nature 285, 542-547.

7. Devereux, J., Haeberi, P., and Smithles, O. (1984). A comprehensive set of sequence analysis programs for the VAX. Nucleic Acids Res. IZ 387-395.

8. Fawcett, J. W. and Keynes, R. J. (1990). Peripheral nerve regeneration. Annu. Rev. Neurosci. 13, 43-60.

9. Fournier, J., Steinberg, R , Gauthier, T., Keane, P. E., Guzzi, U., Coude, F. X., Bougault, I., Maffrand, J. P., Soubrie, P., and Le Fur, G. (1993). Protective effects of SR 57746A in central and peripheral models of neurodegenerative disorders in rodents and primates. Neuroscience 55, 629-641.

10. Funakoshi, H., Frisen, J., Barbany, G., Timmusk, T., Zachrisson, O., Verge, V. M., and Persson, H. (1993). Differential expression of mRNAs for neurotrophins and their receptors after axotomy of the sciatic nerve. J. Cell Biol. 123, 455-465.

11. Giannakopoulos, P., Kovarl, E., French, L. E., Viard, I., Hof, P. R., and Bouras, C. (1998). Possible neuroprotective role of clusterin in Alzheimer's disease: a quantitative immunocytochemical study. Acta Neuropathol. (Berl) 95, 387-394.

12. Grantham, R. (1974). Amino acid difference formula to help explain protein evolution. Science 185, 862-864.

13. Han, B. H., DeMattos, R. B., Dugan, L. L, Kim-Han, J. S., Brendza, R. P., Fryer, J. D., Kierson, M., Cirrito, J., Quick, K., Harmony, J. A, Aronow, B. J., and Holtzman, D. M. (2001). Clusterin contributes to caspase-3-independent brain injury following neonatal hypoxia-ischemia. Nat. Med. 7, 338-343.

14. Hanaoka, Y., Ohl, T., Furukawa, S., Furukawa, Y., Hayashl, K., and Matsukura, S. (1992). Effect of 4-methylcatechol on sciatic nerve growth factor level and motor nerve conduction velocity in experimental diabetic neuropathic process in rats. Exp. Neurol. 115, 292-296.

15. Ho, T. W., McKhann, G. M., and Griffin, J. W. (1998). Human autoimmune neuropathies. Annu Rev Neurosci. 21, 187-226.

16. Jones, K. J. (1993). Recovery from facia paralysis following crush injury of the facial nerve in hamsters: differential effects of gender and androgen exposure. Exp. Neurol. 121, 133-138.

17. Jones, S. E. and Jomary, C. (2002). Clusterin. Int J. Biochem. Cell Biol. 34, 427-431.

18. Kaechi, K, Furukawa, Y., Ikegami, R., Nakemura, N., Omae, F., Hashimoto, Y. Hayashi, K., and Furukawa, S. (1993). Pharmacological induction of physiologically active nerve growth factor in rat peripheral nervous system. J. Pharmacol. Exp. Ther. 264, 321-326.

19. Klotz, L. and Herschom, S. (1998). Early experience with intraoperative cavernous nerve stimulation with penile tumescence monitoring to improve nerve sparing during radical prostatectomy. Urology 52, 537-542.

20. Kounnas, M. Z., Louhnova, E. B , Stefansson, S., Harmony, J. A., Brewer, B. H., Strickland, D. K., and Argraves, W. S. (1995). Identification of glycoprotein 330 as an endocylic receptor for apolipoprotein J/clusterin. J. Biol. Chem. 270, 13070-13075.

21. Kuhn, G., Lie, A., Wilms, S., and Muller, H. W. (1993). Coexpression of PMP22 gene with MBP and P0 during de novo myelination and nerve repair. Glia 8, 256-264.

22. Kwa, M. S., van Schaik, I. N., De Jonge, R. R., Brand, A, Kalaydjieva, L., van Belzen, N., Vermeulen, M., and Baas, F. (2003). Autoimmunoreactivity to Schwann cells in patents with inflammatory neuropathles. Brain 126, 361-375.

23. Lewis, M. E., Neff, N. T., Contreras, P. C., Stong, D. B., Oppenheim, R. W., Grebow, P. E. and Vaught, J. L. (1993). Insulin-like growth factor-1: potential for treatment of motor neuronal disorders. Exp. Neurol. 124, 73-88.

24. McMahon, S. B. and Priestley, J. V. (1995). Peripheral neuropathies and neurotrophic factors: animal models and clinical perspectives. Curr. Opin. Neurobiol. 5, 616-624.

25. Pearson, W. R. (1990), Rapid and sensitive sequence comparison with FASTP and FASTA. Methods Enzymol. 183, 63-98.

26. Pearson, W. R. and Upman, D. J. (1988). Improved tools for biological sequence comparison. Proc. Natl. Acad. Sci. U.S.A 85, 2444-2448.

27. Poon, S., Treweek, T. M., Wilson, M. R., Easterbrook-Smith, S. B., and Carver, J. A. (2002). Clusterin is an extracellular chaperone that specifically interacts with slowly aggregating proteins on their off-folding pathway. FEBS Lett. 513, 259-266.

28. Quinlan, D. M., Epstein, J. I., Carter, B. S., and Walsh, P. C. (199 1a). Sexual function following radical prostatectomy: influence of preservation of neurovascular bundles. J. Urol. 145, 998-1002.

29. Quinlan, D. M., Nelson, R. J., and Walsh, P. C. (1991b). Cavernous nerve grafts restore erectile function in denervated rats. J. Urol. 145, 380-383.

30. Rosenberg, M. E. and Silkensen, J. (1995). Clusterin: physiologic and pathophysiologic considerations. Int. J Biochem Cell Biol 27, 633-445.

31. Shepard, H. M., Leung, D., Stebbing, N., and Goeddel, D. V. (1981). A single amino acid change in IFN-beta1 abolishes its antiviral activity. Nature 294, 563-565.

32. Smith, T. F. and Waterman, M. S. (1981). Identification of common molecular subsequences. J. Mol. Biol. 147, 195-197.

33. Steck, A. J., Schaeren-Wemers, N., and Hartung, H. P. (1998). Demyelinating inflammatory neuropathies, including Guillain-Barré syndrome. Curr Opin Neural. 11, 311-318.

34. Stoppini, L., Buchs, P. A, and Muller, D. (1991). A simple method for organotypic cultures of nervous tissue. J Neurosci. Methods 37, 173-182.

35. Strand, F. L, Lee, S. J., Lee, T. S., Zuccarelli, L. A., Antonawich, F. J., Kume, J., and Williams, K. A. (1993). Non-corticotropic ACTH peptides modulate nerve development and regeneration. Rev. Neurosci. 4, 321-363.

36. Trougakos, I. P. and Gonos, E. S. (2002). Clusterin/apolipoprotein J in human aging and cancer. Int J. Biochem Cell Biol. 34, 1430-1448.

37. Tschopp, J., Chonn, A., Hertig, S., and French, L. E. (1993). Clusterin, the human apolipoprotein and complement inhibitor, binds to complement C7, C8 beta, and the b domain of C9. J. Immunol. 151, 2159-2165.

38. Wehrli, P., Chamay, Y., Vallet, P., Zhu, G. , Harmony, J., Aronow, B., Tschopp, J., Bouras, C., Viard-Leveugle, I., French, L. E., and Giannakopoulos, P. (2001). Inhibition of post-ischemic brain injury by clusterin overexpression. Nat Med 7, 977-979.

1-25. (canceled)

26. A method of preventing or treating a peripheral neurological disease comprising the administration of a composition comprising clusterin, an isoform, mutein, fused protein, functional derivative, active fraction, circularly permutated derivative, or salt thereof, or an agonist of clusterin activity to an individual having peripheral neurological disease.

27. The method according to claim 26, wherein the peripheral neurological disease is selected from the group consisting of traumatic nerve injury of the peripheral nervous system (PNS), demyelinating diseases of the PNS, peripheral neuropathies and peripheral neurodegenerative diseases.

28. The method according to claim 26, wherein the peripheral neurological disease is caused by a congenital metabolic disorder.

29. The method according to claim 27, wherein the peripheral neurological disease is a peripheral neuropathy.

30. The method according to claim 29, wherein the peripheral neuropathy is diabetic neuropathy.

31. The method according to claim 29, wherein the peripheral neuropathy is chemotherapy-induced neuropathy.

32. The method according to claim 26, wherein the clusterin is selected from the group consisting of:
(a) a polypeptide comprising SEQ ID NO: 1;
(b) a polypeptide comprising amino acids 23 to 449 of SEQ ID NO: 1;
(c) a polypeptide comprising amino acids 35 to 449 of SEQ ID NO: 1;
(d) a polypeptide comprising amino acids 23 to 227 of SEQ ID NO: 1;
(e) a polypeptide comprising amino acids 35 to 227 of SEQ ID NO: 1;
(f) a polypeptide comprising amino acids 228 to 449 of SEQ ID NO: 1;
(g) a mutein of any of (a) to (f), wherein the amino acid sequence has at least 40% or 50% or 60% or 70% or 80% or 90% identity to at least one of the sequences in (a) to (f);
(h) a mutein of any of (a) to (f) which is encoded by a DNA sequence which hybridizes to the complement of the native DNA sequence encoding any of (a) to (f) under moderately stringent conditions or under highly stringent conditions;
(i) a mutein of any of (a) to (f) wherein any changes in the amino acid sequence are conservative amino acid substitutions to the amino acid sequences in (a) to (f);
a salt or an isoform, fused protein, functional derivative, active fraction or circularly permutated derivative of any of (a) to (f).

33. The method according to claim 32, wherein the functional derivative comprises a PEG moiety.

34. The method according to claim 34, wherein the fused protein comprises an immunoglobulin (Ig) fusion.

35. The method according to claim 26, wherein the composition further comprises heparin.

36. The method according to claim 26, wherein said composition is simultaneously, sequentially, or separately administered with a composition comprising heparin.

37. The method according to claim 26, wherein the composition further comprises an interferon, osteopontin, or both interfereon and osteopontin, for simultaneous, sequential, or separate use.

38. The method according to claim 37, wherein the interferon is interferon-β.

39. The method according to claim 26, wherein the clusterin is used in an amount of about 0.001 to 100 mg/kg of body weight, or about 1 to 10 mg/kg of body weight, or about 5 mg/kg of body weight.

40. A method of preventing or treating a peripheral neurological disease comprising the administration of a nucleic acid molecule, wherein the nucleic acid molecule encodes:
a) a polypeptide comprising SEQ ID NO: 1;
b) a polypeptide comprising amino acids 23 to 449 of SEQ ID NO: 1;
c) a polypeptide comprising amino acids 35 to 449 of SEQ ID NO: 1;
d) a polypeptide comprising amino acids 23 to 227 of SEQ ID NO: 1;
e) a polypeptide comprising amino acids 35 to 227 of SEQ ID NO: 1;
f) a polypeptide comprising amino acids 228 to 449 of SEQ ID NO: 1;
g) a mutein of any of (a) to (f), wherein the amino acid sequence has at least 40% or 50% or 60% or 70% or 80% or 90% identity to at least one of the sequences in (a) to (e);
h) a mutein of any of (a) to (f) which is encoded by a DNA sequence which hybridizes to the complement of the native DNA sequence encoding any of (a) to (f) under moderately stringent conditions or under highly stringent conditions;
i) a mutein of any of (a) to (f) wherein any changes in the amino acid sequence are conservative amino acid substitutions to the amino acid sequences in (a) to (f); or an isoform, fused protein, functional derivative, active fraction or circularly permutated derivative of any of (a) to (f).

41. The method according to claim 40, wherein the nucleic acid molecule further comprises an expression vector sequence.

42. A method of preventing or treating a peripheral neurological disease comprising the administration of a cell that has been genetically modified to produce clusterin, or an agonist of clusterin activity.

43. A pharmaceutical composition comprising:
a) clusterin, or an agonist of clusterin activity, and heparin, optionally together with one or more pharmaceutically acceptable excipients;
b) clusterin, or an agonist of clusterin activity, and an interferon, optionally together with one or more pharmaceutically acceptable excipients; or
c) clusterin, or an agonist of clusterin activity, and osteopontin, optionally together with one or more pharmaceutically acceptable excipients.


SEQUENCE LISTING (1 sequence)

SEQ ID NO: 1 - protein, 449 aa, homo sapiens
MMKTLLLFVGLLLTWESGQVLGDQTVSDNELQEMSNQGSKYVNKEIQNAVNGVKQIKTLIEKTNEERKTLLSNLEEAKKKKEDALNETRESETKLKELPGVCNETMMALWEECKPCLKQTCMKFYARVCRSGSGLVGRQLEEFLNQSSPFYFWMNGDRIDSLLENDRQQTHMLDVMQDHFSRASSIIDELFQDRFFTREPQDTYHYLPFSLPHRRPHFFFPKSRIVRSLMPFSPYEPLNFHAMFQPFLEMIHEAQQAMDIHFHSPAFQHPPTEFIREGDDDRTVCREIRHNSTGCLRMKDQCDKCREILSVDCSTNNPSQAKLRRELDESLQVAERLTRKYNELLKSYQWKMLNTSSLLEQLNEQFNWVSRLANLTQGEDQYYLRVTTVASHTSDSDVPSGVTEVVVKLFDSDPITVTVPVEVSRKNPKFMETVAEKALQEYRKKHREE